(19) United States
(12) Patent Application Publication
(10) Pub. No.: US 2024/0115686 A1
(43) Pub. Date: Apr. 11, 2024

(54) SHIGELLA MULTI-EPITOPE FUSION ANTIGEN PROTEINS AND METHODS OF USE

(71) Applicant: THE BOARD OF TRUSTEES OF THE UNIVERSITY OF ILLINOIS, Urbana, IL (US)

(72) Inventor: Weiping Zhang, Savoy, IL (US)

(73) Assignee: The Board of Trustees of the University of Illinois, Urbana, IL (US)

(21) Appl. No.: 17/768,766

(22) PCT Filed: Oct. 14, 2020
(86) PCT No.: PCT/US2020/055558

§ 371 Date: Apr. 13, 2022

Related U.S. Application Data
(60) Provisional application No. 62/914,918, filed on Oct. 14, 2019.

(51) Int. Cl. A61K 39/112; A61K 39/108; A61P 31/04; C07K 14/245; C07K 14/25

(52) U.S. Cl. CPC A61K 39/0283; A61K 39/0258; A61P 31/04; C07K 14/245; C07K 14/25; A61K 2039/55544

(57) ABSTRACT

Compositions and methods for eliciting an immune response in a subject (such as against Shigella) are provided. In examples, the compositions can include fusion proteins including one or more epitopes from Shigella or E. coli. In examples, the methods can include administering a disclosed fusion protein to a subject.

### CROSS REFERENCE TO RELATED APPLICATIONS

This claims the benefit of U.S. Provisional Application No. 62/914,918, filed Oct. 14, 2019, which is incorporated herein by reference in its entirety.

### FIELD

This application provides Shigella multi-epitope fusion antigen proteins and compositions and methods for inducing an immune response against Shigella in a subject.

### BACKGROUND

Shigella is a top cause of diarrhea in young children in developing countries (children's diarrhea) as well as children and adults traveling from developed to developing countries (travelers' diarrhea). Antibiotic drugs that have been used to treat Shigella diarrhea (shigellosis) become less effective as Shigella strains increasingly acquire antimicrobial resistance (AMR) or even increase disease progression of Shigella infection. Thus, developing effective vaccines for Shigella is a priority for many public health organizations.

### SUMMARY

Immunological heterogeneity among serotypes is a key obstacle in Shigella vaccine development. The unique epitope- and structure-based multi-epitope fusion antigen (MEFA) immunogens provided herein carry conserved antigenic domains of multiple Shigella virulence determinants and induce cross-protective antibodies against different Shigella species and serotypes, providing a new approach in preparing safe and immunogenic multivalent antigens for development of effective Shigella vaccines.

Disclosed herein are fusion proteins, wherein the fusion protein includes a backbone protein, wherein the backbone protein includes a consensus sequence with at least 90% identity to SEQ ID NO: 4; and at least one heterologous epitope. In example embodiments, the at least one epitope includes 8-15 amino acids. In example embodiments, the at least one epitope includes 10, 11, or 12 amino acids.

In examples, the backbone protein includes at least two peptides from a Shigella virulence factor. In example embodiments, the Shigella virulence factor is IpaD (such as SEQ ID NO: 4). In examples, the at least one heterologous epitope includes a peptide of a Shigella virulence factor. In example embodiments, the Shigella virulence factor includes one or more of IpaD, IpaB, VirG, GuaB, StxA, Stx2A, and StxB. In example embodiments, the at least one heterologous epitope includes one or more of SEQ ID NOs: 10, 12, 16, 18, 20, 22, and 24. In examples, the fusion protein disclosed herein can further include at least one homologous epitope (such as one or more of SEQ ID NO: 6, 8, and 14). The heterologous and homologous (if included) epitopes may be contiguous or non-contiguous with one another.

In specific, non-limiting examples, the fusion protein can include a backbone protein, wherein the backbone protein includes a consensus sequence with at least 90% identity to SEQ ID NO: 4; and at least one epitope, wherein the at least one epitope includes at least one heterologous epitope and at least one homologous epitope, wherein the at least one heterologous epitope includes each of SEQ ID NOS: 10, 12, 16, 18, 20, 22, and 24 and the at least one homologous epitope includes each of SEQ ID NOS: 6, 8, and 14. In some examples, the fusion protein can include SEQ ID NO: 2. The heterologous and homologous epitopes may be contiguous or non-contiguous with one another.

Disclosed herein are nucleic acids encoding the fusion proteins disclosed herein. In examples, the nucleic acid encoding the backbone protein includes a consensus sequence at least 90% identical to SEQ ID NO: 3. In example embodiments, the nucleic acid encoding the backbone protein includes SEQ ID NO: 3. In examples, the nucleic acid encoding at least one heterologous epitope includes a nucleic acid encoding a peptide of a Shigella virulence factor (such as one or more of IpaD, IpaB, VirG, GuaB, StxA, Stx2A, and StxB). In example embodiments, the nucleic acid encoding at least one heterologous epitope includes one or more of SEQ ID NO: 9, 11, 15, 17, 19, 21, and 23. In examples, the nucleic acids can also encode at least one homologous epitope (such as one or more of SEQ ID NO: 5, 7, and 13). In specific, non-limiting examples, the nucleic acids include SEQ ID NO: 1.

Disclosed herein are pharmaceutical compositions, which can include a pharmaceutically acceptable carrier and any of the fusion proteins or nucleic acids disclosed herein. In some examples, the pharmaceutical compositions can also include an adjuvant. Further disclosed herein are vectors comprising the nucleic acids disclosed herein. In example embodiments, the vectors further include at least one nucleic acid that encodes at least one peptide of Escherichia coli or Vibrio cholera, such as in an additional fusion protein (for example, with at least two epitopes, such as ETEC antigens CFA/I/II/IV MEFA (e.g., SEQ ID NO: 26), and/or toxoid fusion 3xSTa_{N12S}-mnLT_{R192G/L211A} (e.g., SEQ ID NO: 28)). Also disclosed herein are isolated host cells transformed with any of the vectors disclosed herein.

Disclosed herein are immunogenic compositions that include any of the fusion proteins, nucleic acids, pharmaceutical compositions, or vectors disclosed herein. For example, the immunogenic composition can include a fusion protein disclosed herein and an additional fusion protein (such as ETEC antigens CFA/I/II/IV MEFA and/or toxoid fusion 3xSTa_{N12S}-mnLT_{R192G/L211A}). In example embodiments of the immunogenic compositions, any of the fusion proteins disclosed herein can be expressed in Ty21a.

Disclosed herein are methods of inducing an immune response (such as a protective response) in a subject (such as a human subject) that can include administering (such as subcutaneously (SC), intramuscularly (IM), or intradermally (ID)) an effective amount of any of the fusion proteins, immunogenic compositions, nucleic acids, vectors, or pharmaceutical compositions disclosed herein. In examples, the methods can include selecting a subject in or traveling to an area with endemic Shigella (such as S. flexneri, S. sonnei, S. dysenteriae, and/or S. boydii) or Escherichia coli (such as enterotoxigenic E. coli) and/or a subject that does not have a Shigella (such as S. flexneri, S. sonnei, S. dysenteriae, and/or S. boydii) or E. coli (such as enterotoxigenic E. coli) infection. In examples, the subject is a child at or less than 5 years old, a child at or less than 1 year old, a child greater than 5 years old, or an adult.

The foregoing and other features of the disclosure will become more apparent from the following detailed description, which proceeds with reference to the accompanying figures.

### BRIEF DESCRIPTION OF THE DRAWINGS

FIGS. 1A-1C: Shigella MEFA (9745) genetic structure diagram (FIG. 1A), protein modeling (FIG. 1B), and characterization in SDS-PAGE with Coomassie blue staining or Western blot with anti-IpaD mouse anti-serum (FIG. 1C).

FIGS. 2A-2B: Serum IgG titers to IpaB, IpaD, VirG, GuaB, StxA, Stx2A and StxB in the mice SC immunized with Shigella MEFA (FIG. 2A). Mouse serum antibody invasion inhibition of Shigella species and important serotypes in HeLa cells (FIG. 2B). The 100% reference refers to the number of bacteria invaded in cells incubated with a control serum sample.

FIG. 3: Serum IgG titers in the serum samples of mice IM immunized with Shigella MEFA protein with or without dmLT adjuvant.

FIGS. 4A-4E: Shigella MEFA-induced antibody neutralization of Shiga toxin cytotoxicity. (FIG. 4A) Vero cells in cell culture medium; (FIG. 4B) Vero cells incubated with Shiga toxin, 50 11.1 filtrates of Shigella dysenteriae type 1 (9786) to show CD50. (FIG. 4C) Vero cells incubated with Shiga toxin pre-treated with the control mouse serum (1:24). (FIG. 4D) Vero cells incubated with Shiga toxin pre-treated with the mouse serum (1:24) of the group IM immunized with Shigella MEFA, w/dmLT. (FIG. 4E) Vero cells incubated with Shiga toxin pre-treated with the mouse serum (1:192) of the group IM immunized with Shigella MEFA, w/dmLT.

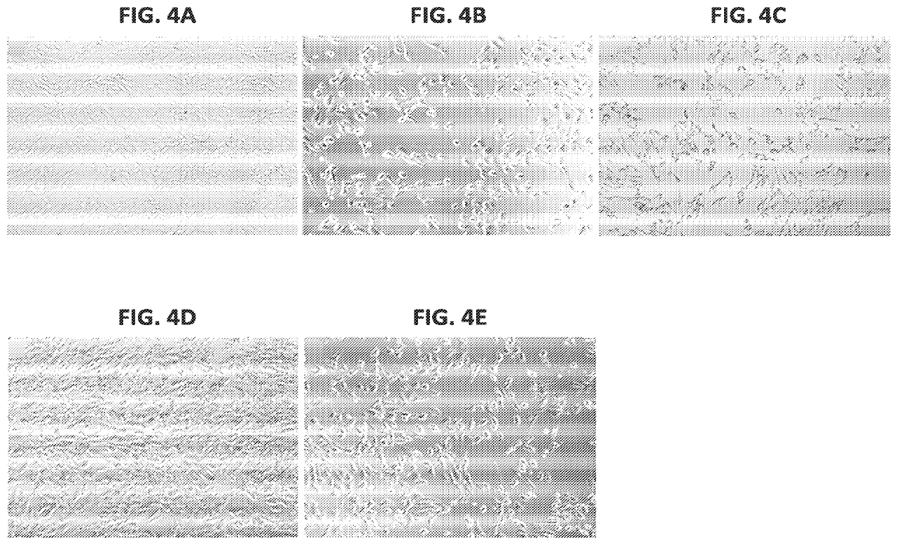

FIG. 5: Mouse serum IgG antibody titers (log₁₀) from the group IM co-administered with Shigella MEFA and ETEC toxoid fusion 3xSTa_{N12S}-mnLT_{R192G/L211A}. Double mutant heat-labile toxin (dmLT) was used as the adjuvant in mouse immunization.

FIG. 6: Mouse serum IgG antibody titers (log₁₀) from the group IM co-administered with Shigella MEFA and ETEC adhesin MEFA CFA/FIT/IV. Double mutant heat-labile toxin (dmLT) was used as the adjuvant in mouse immunization.

FIG. 7: Mouse serum IgG antibody titers (log₁₀) from the group IM co-administered with Shigella MEFA, ETEC adhesin MEFA CFA/FIT/IV, and ETEC toxoid fusion 3XSTa_{N12S}-mnLT_{R192G/L211A}. Double mutant heat-labile toxin (dmLT) was used as the adjuvant in mouse immunization.

FIGS. 8A-8B: Mouse serum antibodies derived from co-administration of Shigella MEFA (S MEFA) and ETEC toxoid fusion 3xSTa_{N12S}-mnLT_{R192G/L211A} (Toxoid) neutralized STa toxin and CT toxin from cyclic cGMP and cAMP ELISAs. (FIG. 8A) Anti-mouse serum antibodies prevented STa toxin from elevating intracellular cGMP levels. (FIG. 8B) Anti-mouse serum antibodies prevented CT (cholera toxin) toxin from elevating intracellular cAMP levels. dmLT is the adjuvant used in mouse immunization.

### SEQUENCE LISTING

Any nucleic acid and amino acid sequences listed herein or in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is an exemplary nucleic acid sequence encoding a fusion protein with a Shigella IpaD backbone protein and homologous and heterologous epitopes from Shigella virulence factors IpaD, IpaB, VirG, GuaB, StxA, Stx2A, and StxB.

SEQ ID NO: 2 is an exemplary fusion protein with a Shigella IpaD backbone protein and homologous and heterologous epitopes from Shigella virulence factors IpaD, IpaB, VirG, GuaB, StxA, Stx2A, and StxB.

SEQ ID NO: 3 is an exemplary consensus nucleic acid sequence that encodes a backbone protein, where ‘N’ indicates a location for insertion or substitution with a heterologous epitope or where a homologous epitope is located.

SEQ ID NO: 4 is an exemplary consensus amino acid sequence for backbone protein, where ‘X’ indicates a location for insertion or substitution with a heterologous epitope or where a homologous epitope is located.

SEQ ID NO: 5 is an exemplary nucleic acid sequence encoding an IpaD epitope.

SEQ ID NO: 6 is an exemplary IpaD epitope amino acid sequence.

SEQ ID NO: 7 is an exemplary nucleic acid sequence encoding an IpaD epitope.

SEQ ID NO: 8 is an exemplary IpaD epitope amino acid sequence.

SEQ ID NO: 9 is an exemplary nucleic acid sequence encoding an IpaB epitope.

SEQ ID NO: 10 is an exemplary IpaB epitope amino acid sequence.

SEQ ID NO: 11 is an exemplary nucleic acid sequence encoding an IpaB epitope.

SEQ ID NO: 12 is an exemplary IpaB epitope amino acid sequence.

SEQ ID NO: 13 is an exemplary nucleic acid sequence encoding an IpaD epitope.

SEQ ID NO: 14 is an exemplary IpaD epitope amino acid sequence.

SEQ ID NO: 15 is an exemplary nucleic acid sequence encoding a VirG epitope.

SEQ ID NO: 16 is an exemplary VirG epitope amino acid sequence.

SEQ ID NO: 17 is an exemplary nucleic acid sequence encoding a StxA epitope.

SEQ ID NO: 18 is an exemplary StxA epitope amino acid sequence.

SEQ ID NO: 19 is an exemplary nucleic acid sequence encoding a GuaB epitope.

SEQ ID NO: 20 is an exemplary GuaB epitope amino acid sequence.

SEQ ID NO: 21 is an exemplary nucleic acid sequence encoding a Stx2 epitope.

SEQ ID NO: 22 is an exemplary Stx2 epitope amino acid sequence.

SEQ ID NO: 23 is an exemplary nucleic acid sequence encoding a StxB epitope.

SEQ ID NO: 24 is an exemplary StxB epitope amino acid sequence.

SEQ ID NO: 25 is an exemplary nucleic acid encoding a fusion protein with ETEC antigens.

SEQ ID NO: 26 is an exemplary amino acid sequence of a fusion protein with ETEC antigens.

SEQ ID NO: 27 is an exemplary nucleic acid encoding a fusion protein with ETEC antigens.

SEQ ID NO: 28 is an exemplary amino acid sequence of a fusion protein with ETEC antigens.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising an epitope” includes single or plural epitopes and is considered equivalent to the phrase “comprising at least one epitope.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® Accession Nos. referred to herein are the sequences available on Oct. 14, 2019. All references, including journal articles, patents, and patent publications, and GenBank® Accession numbers cited herein are incorporated by reference in their entirety.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided.

Adjuvant: A substance or vehicle that non-specifically enhances the immune response to an antigen (for example, a Shigella or Escherichia coli antigen). Adjuvants can be used with the compositions disclosed herein, for example, as part of a Shigella immunogenic composition provided herein. Adjuvants can include a suspension of minerals (alum, aluminum hydroxide, or phosphate) on which antigen is adsorbed or a water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants may also include biological molecules, such as costimulatory molecules. Exemplary biological adjuvants include IL-2, RANTES, GM-CSF, TNF-α, IFN-γ, G-CSF, LFA-3, CD72, B7-1, B7-2, OX-40L and 41 BBL. In one example the adjuvant is one or more toll-like receptor (TLR) agonists, such as an agonist of TLR1/2 (which can be a synthetic ligand) (for example, Pam3Cys), TLR2 (for example, CFA, Pam2Cys), TLR3 (for example, polyI:C, poly A:U), TLR4 (for example, MPLA, Lipid A, and LPS), TLR5 (for example, flagellin), TLR7 (for example, gardiquimod, imiquimod, loxoribine, Resiquimod®), TLR7/8 (for example, R0848), TLR8 (for example, imidazoquionolines, ssPolyU, 3M-012), TLR9 (for example, ODN 1826 (type B), ODN 2216 (type A), CpG oligonucleotides) and/or TLR11/12 (for example, profilin). In one example, the adjuvant is lipid A, such as lipid A monophosphoryl (MPL) from Salmonella enterica serotype Minnesota Re 595 (for example, Sigma Aldrich Catalog #L6895). In another example the adjuvant is an enterotoxin based adjuvant, such as double mutant heat-labile toxin (dmLT).

Administer: As used herein, administering a composition (such as a Shigella or E. coli protein composition) to a subject means to give, apply or bring the composition into contact with the subject. Administration can be accomplished by any of a number of routes, such as, for example, intramuscular, intranasal, pulmonary, topical, oral, subcutaneous, intraperitoneal, intravenous, intrathecal, rectal, vaginal, or intradermal. In specific examples, administration is intramuscular or subcutaneous.

Antigen or immunogen: A compound, composition, or substance that can stimulate the production of an immune response in a subject, including compositions that are injected or absorbed into a subject. An antigen reacts with the products of specific humoral or cellular immunity, including those induced by heterologous immunogens.

Attenuated: In the context of the type of live pathogen, the pathogen (for example, a bacterial pathogen, such as Ty21a) is attenuated if its ability to produce disease is reduced (or even eliminated) compared with a wild-type pathogen.

Escherichia coli: E. coli is a Gram-negative, facultative anaerobic, rod-shaped, coliform bacterium commonly found in the lower intestine of warm-blooded organisms (endotherms). Virulent strains can cause serious disease. In specific examples, the E. coli is enterotoxigenic E. coli (ETEC), which is the most common cause of traveler's diarrhea with 840 million cases worldwide in developing countries each year. The bacteria are typically transmitted through contaminated food or drinking water, adhere to the intestinal lining, where they secrete enterotoxins, leading to watery diarrhea. The rate and severity of infections are higher among children under the age of five, which include 380,000 deaths annually. Administration of antibiotics has been shown to shorten the course of illness and duration of excretion of ETEC in adults in endemic areas and in traveler's diarrhea, but the rate of resistance to commonly used antibiotics is increasing and they are generally not recommended. The antibiotic used depends upon susceptibility patterns in the particular geographical region; the antibiotics typically selected for administration include fluoroquinolones, azithromycin, and rifaximin.

Fusion protein: A protein containing amino acid sequence from at least two different (heterologous) proteins or peptides. In some examples herein, the fusion protein comprises a backbone protein and one or more heterologous peptides, such as one or more heterologous epitopes. The backbone and heterologous sequences may be contiguous or non-contiguous. Fusion proteins can be generated, for example, by expression of a nucleic acid sequence engineered from nucleic acid sequences encoding at least a portion of two different (heterologous) proteins. To create a fusion protein, the nucleic acid sequences must be in the same reading frame and contain no internal stop codons. Fusion proteins, particularly short fusion proteins, can also be generated by chemical synthesis.

Heterologous: A heterologous protein or nucleic acid refers to a protein or nucleic acid derived from a different source (such as a peptide or epitope from a different protein) or species.

Immune response: A response of a cell of the immune system, such as a B-cell, T-cell, macrophage, or polymorphonucleocyte, to a stimulus such as an antigen/immunogen or vaccine (such as a Shigella or E. coli immunogenic composition or vaccine). An immune response can include any cell of the body involved in a host defense response, including for example, an epithelial cell that secretes an interferon or a cytokine. An immune response includes, but is not limited to, an innate immune response. As used herein, a protective immune response refers to an immune response that protects a subject from infection (prevents infection or prevents the development of disease associated with infection). Methods of measuring immune responses include, for example, measuring proliferation and/or activity of lymphocytes (such as B or T cells), secretion of cytokines or chemokines, inflammation, antibody production and the like.

Isolated: An “isolated” biological component (such as a nucleic acid, protein, or cell) has been substantially separated or purified away from other biological components (such as cell debris, or other proteins or nucleic acids). Biological components that have been “isolated” include those components purified by standard purification methods. The term also embraces recombinant nucleic acids and proteins and chemically synthesized nucleic acids or peptides.

Modification: A change in a nucleic acid or protein sequence. For example, sequence modifications include substitutions, insertions, and deletions, or combinations thereof. For proteins, insertions include amino and/or carboxyl terminal fusions as well as intrasequence insertions of single or multiple amino acid residues. Insertions for nucleic acid sequence include 5′ or 3′ additions or intrasequence insertions of single or multiple nucleotides. Deletions are characterized by the removal of one or more amino acid residues from a protein sequence or one or more nucleotides from a nucleic acid sequence. Substitutions are those in which at least one amino acid residue or nucleotide has been removed and a different residue or nucleotide inserted in its place. Substitutions, deletions, insertions or any combination thereof may be combined to arrive at a final modified sequence. Protein modifications can be prepared by modification of nucleotides in the DNA encoding the protein, thereby producing DNA encoding the modification. Techniques for making insertion, deletion and substitution mutations at predetermined sites in DNA or RNA having a known sequence are well known in the art. A “modified” protein, nucleic acid or virus is one that has one or more modifications as outlined above.

Nucleic acid: A polymeric form of nucleotides, which may include both sense and anti-sense strands of RNA, cDNA, genomic DNA, genomic RNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (such as rRNA, tRNA and mRNA) or a defined sequence of amino acids and the biological properties resulting therefrom.

ORF (open reading frame): A series of nucleotide triplets (codons) coding for amino acids without any termination codons. These sequences are usually translatable into a peptide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: Pharmaceutically acceptable carriers are known. Remington's Pharmaceutical Sciences, by E. W. Martin, Mack Publishing Co., Easton, PA, 22nd Edition, 2013, describes compositions and formulations suitable for pharmaceutical delivery of the disclosed compositions.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), non-toxic solid carriers can include, for example, pharmaceutical grades of mannitol, lactose, starch, or magnesium stearate. In addition to biologically neutral carriers, pharmaceutical compositions (such as immunogenic compositions) to be administered can contain minor amounts of non-toxic auxiliary substances, such as wetting or emulsifying agents, preservatives, and pH buffering agents and the like, for example sodium acetate or sorbitan monolaurate. In particular embodiments, in compositions suitable for administration to a subject, the carrier may be sterile and/or suspended or otherwise contained in a unit dosage form containing one or more measured doses of the composition suitable to induce the desired immune response. The unit dosage form may be, for example, in a sealed vial that contains sterile contents or a syringe for injection into a subject, or lyophilized for subsequent solubilization and administration or in a solid or controlled release dosage.

Peptide: Any chain of amino acids, regardless of length or post-translational modification (for example, glycosylation or phosphorylation). “Peptide” applies to amino acid polymers including naturally occurring amino acid polymers and non-naturally occurring amino acid polymers, including such polymers in which one or more amino acid residues is a non-natural amino acid, for example, an artificial chemical mimetic of a corresponding naturally occurring amino acid. A “residue” refers to an amino acid or amino acid mimetic incorporated in a peptide by an amide bond or amide bond mimetic. A peptide has an amino terminal (N-terminal) end and a carboxy terminal (C-terminal) end.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Recombinant: A recombinant nucleic acid molecule is one that has a sequence that is not naturally occurring, for example, includes one or more nucleic acid substitutions, deletions, or insertions, and/or has a sequence that is made by an artificial combination of two otherwise separated segments of sequence. This artificial combination can be accomplished by chemical synthesis or by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In some embodiments, a recombinant protein includes a fusion protein, for example, a protein that includes one or more heterologous peptides (such as epitopes).

Shigella: Shigella is a genus of Gram-negative, facultative aerobic, non-spore-forming, nonmotile, rod-shaped bacteria genetically closely related to E. coli that causes human shigellosis (Shigella infection) as well as disease in primates. Shigella infection causes diarrhea, including moderate-to-severe diarrhea in children, worldwide with about 80-165 million cases and 74,000-600,000 deaths annually. Infection also typically causes dysentery. Shigella includes S. dysenteriae (serogroup A, 15 serotypes), S. flexneri (serogroup B, six serotypes), S. boydii (serogroup C, 19 serotypes), and S. sonnei (serogroup D, one serotype). Each Shigella genome includes a virulence plasmid that encodes conserved primary virulence determinants. The Shigella chromosomes share most of their genes with E. coli K12 strain MG1655.

Shigella infection is typically caused by ingestion of bacteria, with the Shigella invading the epithelial lining of the colon, causing severe inflammation and death of the cells lining the colon, which results in diarrhea and dysentery. Some Shigella strains also produce toxins that contribute to disease during infection, such as S. flexneri (ShET1 and ShET2) and S. dysenteriae (Shiga toxin, which is associated with potentially fatal hemolytic-uremic syndrome).

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a given gene or protein will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989; Corpet et al., Nucleic Acids Research 16:10881-10890, 1988; and Pearson and Lipman, Proc. Natl. Acad. Sci. U.S.A. 85:2444, 1988. Altschul et al., Nature Genet. 6:119-129, 1994.

The NCBI Basic Local Alignment Search Tool (BLAST™) (Altschul et al., J. Mol. Biol. 215:403-410, 1990) is available from several sources, including the National Center for Biotechnology Information (NCBI, Bethesda, MD) and on the Internet, for use in connection with the sequence analysis programs blastp, blastn, blastx, tblastn and tblastx.

Subject: Living multi-cellular vertebrate organisms, a category that includes both human and non-human mammals. In some examples, a subject is one that can be infected with Shigella or E. coli, such as humans.

Synthetic: Produced by artificial means, for example a synthetic nucleic acid or protein can be chemically synthesized in a laboratory.

Therapeutically effective amount (or effective amount): The amount of agent, such as a disclosed fusion protein, that is sufficient to induce a response, such as an immune response or is sufficient to treat, reduce, and/or ameliorate the symptoms and/or underlying causes of a disorder or disease, for example, to induce an immune response to Shigella or E. coli or to inhibit and/or treat Shigella or E. coli infection and/or disease resulting therefrom (such as Shigellosis). In some embodiments, a therapeutically effective amount is sufficient to reduce or eliminate a symptom of a disease. For instance, this can be the amount necessary to inhibit pathogen (such as Shigella or E. coli) replication or to measurably alter outward symptoms of pathogen infection.

In one example, a desired response is to inhibit or reduce Shigella or E. coli infection. Shigella or E. coli infection does not need to be completely eliminated for the method to be effective. For example, administration of a therapeutically effective amount of the agent can decrease the Shigella or E. coli infection (for example, as measured by infection of cells, or by number or percentage of subjects infected by Shigella or E. coli) by a desired amount, for example by at least 50%, at least 60%, at least 70%, at least 80%, at least 90%, at least 95%, at least 98%, or even at least 99% compared to a suitable control.

It is understood that producing a protective immune response against a pathogen can require multiple administrations of the immunogenic composition. Thus, a therapeutically effective amount encompasses a fractional dose that contributes in combination with previous or subsequent administrations to attaining a protective immune response. For example, a therapeutically effective amount of an agent can be administered in a single dose, or in several doses, during a course of treatment (such as a prime-boost vaccination treatment). However, the therapeutically effective amount and timing of administration can depend on the subject being treated, the severity and type of the condition being treated, and the manner of administration. A unit dosage form of the agent can be packaged in a therapeutic amount, or in multiples of the therapeutic amount, for example, in a vial (such as with a pierceable lid) or syringe having sterile components.

Vaccine: A preparation of immunogenic material capable of stimulating an immune response. The immunogenic material may include attenuated or killed microorganisms (such as attenuated viruses) or antigenic proteins, peptides, or nucleic acids encoding an antigen. Vaccines may elicit both prophylactic and therapeutic responses. Methods of administration vary according to the vaccine, but may include inoculation, ingestion, inhalation, or other forms of administration. Inoculations can be delivered by any of a number of routes, including parenteral, such as intravenous, subcutaneous, or intramuscular. In specific embodiments, vaccines can be administered via a subcutaneous or intramuscular route. Vaccines may be administered with an adjuvant to increase the immune response to the vaccine.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic nucleic acids and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like. A vector can be a viral vector.

Virulence factors: Virulence factors enable a host to replicate and disseminate bacteria within a host in part by subverting or eluding host defenses. In example embodiments, virulence factors include Shigella virulence factors, such as IpaD (activates bacterial secretion system that secretes proteins bacteria use to invade a host), IpaB (forms translocon pore for exporting virulence factors into host cells), VirG (also known as IcsA, forms adhesions with host epithelial cells), GuaB (facilitates nucleic acid synthesis), and Shiga toxins (such as StxA, Stx2A, and StxB, which facilitate entry into a host cell and arrest of host cell protein synthesis).

Overview

A key challenge in Shigella vaccine development is the heterogeneity among Shigella species and serotypes. As disclosed herein, a multivalent Shigella immunogen was constructed by applying a novel epitope- and structure-based MEFA (multi-epitope fusion antigen) vaccinology platform and using Shigella invasion plasmid antigen IpaD as a backbone to present conserved immunodominant epitopes from IpaB, IpaD, VirG, GuaB, and Shiga toxins. Mice immunized with the disclosed Shigella MEFA protein developed robust IgG responses to IpaB, IpaD, VirG, GuaB, Stx1, and Stx2 toxins, and importantly the sera of the immunized mice inhibited invasion of S. sonnei, S. flexneri serotypes 2a, 3, 6, S. boydii, and S. dysenteriae type 1 bacteria to HeLa cells and neutralized cytotoxicity of Shiga toxins in vitro. These results indicate that the Shigella MEFA protein is broadly immunogenic and induces antibodies cross protective against four Shigella species and the important serotypes, demonstrating the applicability of this protein in development of a broadly protective vaccine against Shigella infections.

Fusion Protein

Methods and compositions are disclosed herein for a fusion protein that includes a backbone protein and at least one epitope from a Shigella protein, such as a Shigella virulence factor. The methods described herein include administering the fusion protein in a pharmaceutical composition to a subject (such as subcutaneously or intramuscularly). In some embodiments, the at least one epitope includes at least one heterologous epitope (such as an epitope from a different protein from the backbone protein). Homologous epitopes (such as an epitope from the backbone protein) can also be included.

### Backbone Protein

The disclosed fusion proteins include at least one backbone protein (one or more backbone proteins can be combined into one fusion protein, for example, using a linker, such as 1, 2, 3, 4, or 5 backbone proteins). In example embodiments, the at least one backbone protein allows the fusion protein to maintain a tertiary or quaternary structure while presenting one or more epitopes (such as at least one heterologous epitope and/or homologous epitope). In example embodiments, the backbone protein includes a series of peptides from a single protein or protein fold. For example, a backbone protein can include the same or similar tertiary or quaternary structural features as a single protein or protein fold. Example structural features that a backbone protein can retain from a single protein or protein fold include solvent inaccessible amino acids and interactions, salt bridges, disulfide bonds, secondary structure, and/or protein fold. In some examples, the backbone protein is derived from a Shigella virulence factor. In specific examples, the backbone protein is derived from Shigella virulence factor IpaD.

Exemplary backbone sequences include the following: ATGGGCAATATAACAACTCTGACTAATAGTATTTCCACCTCATCATTCNNNNNNNNNN NNNNNNNNNNNNNNNNNNNNNNNNNNNGTTAATTCTGATATAAAAACAACGACCAGTT CTCATCCTGTAAGTTCCCTTACTATGCTCAACGACACCCTTCATAATATCAGAACAACA AATCAGGCATTAAAGAAAGATCTCTCACAAAAAACGTTGACTAAAACATCGCTAGAA GAAATAGCATTACATTCATCTCAGATTAGCATGGATGTAAACAAATCCGCTCAACTAT TGGATATTCTTNNNNNNNNNNNNNNNNNNNNNNNNNNNNNNNGCAAGAGATTCAC ATTCAGCNNNNNNNNNNNNNNNNNNNNNNNNNNNNNNATGATATCTCACAGAGAA CTGTGGGATAAAATTGCAAAGTCAATCAATAATATTAATGAACAGTATCTGAAAGTAT ATGAACATGCCGTTAGTTCATATACTCAANNNNNNNNNNNNNNNNNNNNNNNNNNN NNNNNCTTGCCGGCTGGATCNNNNNNNNNNNNNNNNNNNNNNNNNNNNNAAATTA CAAGTCAAGTCTCTTAAANNNNNNNNNNNNNNNNNNNNNNNNNNNNNNAAAGATAA ACCGCTATATCCAGCAAATAATACTGTTNNNNNNNNNNNNNNNNNNNNNNNNNNNN NNNNNNNGAATTAGGTGGAACANNNNNNNNNNNNNNNNNNNNNNNNNNNNNNTATG TTGTCAATATAAACATGACCCCAATAGACAATATGNNNNNNNNNNNNNNNNNNNNN NNNNNNNNNNCCGGTGAGGTTGTGCTAGATAATGCAAAATATCAGGCATGGAATG CCGGANNNNNNNNNNNNNNNNNNNNNNNNNNNNNAATCTTCAAACCTTAGTTCAAA AATACAGTAATGCCAATAGTATTTTTGATAATTTAGTAAAGGTTTTGAGTAGTACAATA AGTTCATGTACAGATACAGATAAACTTTTTCTCCATTTCTGA (SEQ ID NO: 3, an exemplary consensus nucleic acid sequence that encodes a backbone protein, where ‘N’ indicates a location for insertion or substitution with a heterologous epitope or where a homologous epitope is located).

The number of nucleotides (‘N’) at each epitope site can vary, depending on the length of the inserted epitope. In specific, non-limiting examples, the backbone nucleic acid, includes a consensus sequence with at least 90% identity (such as at least 91%, at least 92%, at least 93%, at least 94%, at least 95%, at least 96%, at least 97%, at least 98%, at least 99%, or at least 100% identity) to SEQ ID NO: 3. The consensus sequence (or the part of the sequence denoted with a specific nucleotide) denotes the portion that is considered the backbone, whereas ‘N’ can vary and may include an epitope sequence (such as a heterologous or homologous sequence).

MGNITTLTNSISTSSFXXXXXXXXXXXXVNSDIKTTTSSHPVSSLTMLNDTLHNIRTTNQAL KKDLSQKTLTKTSLEEIALHSSQISMDVNKSAQLLDILXXXXXXXXXXARELLHSAXXXX XXXXXXMISHRELWDKIAKSINNINEQYLKVYEHAVSSYTQXXXXXXXXXXXLAGWIXX XXXXXXXXKLQVKSLKXXXXXXXXXXKDKPLYPANNTVXXXXXXXXXXXXELGGTXX XXXXXXXXYVVNINMTPIDNMXXXXXXXXXXXXGEVVLDNAKYQAWNAGXXXXXXX XXXNLQTLVQKYSNANSIFDNLVKVLSSTISSCTDTDKLFLHF (SEQ ID NO: 4, an exemplary consensus amino acid sequence for backbone protein, where ‘X’ indicates a location for insertion or substitution with a heterologous epitope or where a homologous epitope is located).

The number of amino acids (‘X’) at each epitope site can vary, depending on the length of the inserted epitope. In specific, non-limiting examples, the backbone protein, includes a consensus sequence with at least 90% identity (such as at least 91%, at least 92%, at least 93%, at least 94%, at least 95%, at least 96%, at least 97%, at least 98%, at least 99%, or at least 100% identity) to SEQ ID NO: 4. The consensus sequence (or the part of the sequence denoted with a specific amino acid) denotes the portion that is considered the backbone, whereas ‘X’ can vary and may include an epitope sequence (such as a heterologous or homologous sequence).

### Epitopes

The disclosed fusion proteins include at least one heterologous epitope. In some examples, homologous epitopes are also included. Example epitopes (such as heterologous or homologues epitopes) can include various sizes. For example, epitope sizes can range from 5-30 amino acids, including ranges of about 5-10, 8-15, 8-20, 10-12, 10-15, 10-20, and 10-30 amino acids, for example, at least about 5, 7, 8, 9, 10, 11, 12, 13, 14, 15, 18, 20, 25, or 30 amino acids in length, or about 10, 11, or 12 amino acids long. Nucleic acids that encode the epitopes are also included and can range in size from about 15-90 nucleotides, including ranges of about 15-30, 24-45, 24-60, 30-36, 30-45, 30-60, and 30-90 nucleotides, for example, at least about 15, 21, 24, 27, 30, 33, 36, 39, 42, 45, 54, 60, 75, or 90 nucleotides in length, or about 30, 33, or 36 nucleotides long. The epitopes may be contiguous or non-contiguous with one another in the backbone protein. In some examples, all of the epitopes are non-contiguous. In other examples, one or more of the epitopes are contiguous, while one or more other epitopes are non-contiguous with the other epitopes.

In some embodiments, the epitope can be, for example, one or more peptides derived from one or more virulence factors, such as a Shigella virulence factor (for example, IpaD, IpaB, VirG, GuaB, StxA, Stx2, and/or StxB). In some embodiments, the backbone protein includes one or more peptides (such as 1, 2, 3, 4, 5, 6, 7, 8, 9, 10, or more peptides) from at least one virulence factor, such as at least one Shigella virulence factor. Thus, in specific, non-limiting examples, where the backbone protein is derived from IpaD, one or more homologous epitopes from IpaD can be included, and heterologous epitopes from one or more additional virulence factors, such as IpaB, VirG, GuaB, StxA, Stx2A, and/or StxB can be included. Exemplary epitopes are provided herein; however, a person of skill in the art can readily identify additional epitopes of use.

Exemplary epitope sequences include the following:

AGCCCAAACAATACCAACGGTTCATCAACCGAAACA (SEQ ID NO: 5, an exemplary nucleic acid sequence encoding an IpaD epitope, such as a homologous epitope where the backbone protein is derived from IpaD)

SPNNTNGSSTET (SEQ ID NO: 6, an exemplary IpaD epitope amino acid sequence, such as a homologous epitope where the backbone protein is derived from IpaD)

TCCAAGAAAGAATATCCAATTAATAAAGAC (SEQ ID NO: 7 an exemplary nucleic acid sequence encoding an IpaD epitope, such as a homologous epitope where the backbone protein is derived from IpaD)

SKKEYPINKD (SEQ ID NO: 8, an exemplary IpaD epitope amino acid sequence, such as a homologous epitope where the backbone protein is derived from IpaD)

CAACTAGTTGGAAAAAATAATGAAGAATCT (SEQ ID NO: 9, an exemplary nucleic acid sequence encoding an IpaB epitope, such as a heterologous epitope where the backbone protein is derived from IpaD)

QLVGKNNEES (SEQ ID NO: 10, an exemplary IpaB epitope amino acid sequence, such as a heterologous epitope where the backbone protein is derived from IpaD)

TCTGCTGAACAGCTATCAACCCAGCAGAAAAGT (SEQ ID NO: 11, an exemplary nucleic acid sequence encoding an IpaB epitope, such as a heterologous epitope where the backbone protein is derived from IpaD)

SAEQLSTQQKS (SEQ ID NO: 12, an exemplary IpaB epitope amino acid sequence, such as a heterologous epitope where the backbone protein is derived from IpaD)

TCTCCCGGAGGTAACGACGGAAACTCCGTG (SEQ ID NO: 13, an exemplary nucleic acid sequence encoding an IpaD epitope, such as a homologous epitope where the backbone protein is derived from IpaD)

SPGGNDGNSV (SEQ ID NO: 14, an exemplary IpaD epitope amino acid sequence, such as a homologous epitope where the backbone protein is derived from IpaD)

AGTGATAGTGATGGGGGGAATGGAGGTGAT (SEQ ID NO: 15, an exemplary nucleic acid sequence encoding a VirG epitope, such as a heterologous epitope where the backbone protein is derived from IpaD)

SDSDGGNGGD (SEQ ID NO: 16, an exemplary VirG epitope amino acid sequence, such as a heterologous epitope where the backbone protein is derived from IpaD)

CTGCCTGACTATCATGGACAAGACTCTGTTCGTGTA (SEQ ID NO: 17, an exemplary nucleic acid sequence encoding a StxA epitope, such as a heterologous epitope where the backbone protein is derived from IpaD)

LPDYHGQDSVRV (SEQ ID NO: 18, an exemplary StxA epitope amino acid sequence, such as a heterologous epitope where the backbone protein is derived from IpaD)

GAACGTAAACCGAACGCTTGTAAAGACGAG (SEQ ID NO: 19, an exemplary nucleic acid sequence encoding a GuaB epitope, such as a heterologous epitope where the backbone protein is derived from IpaD)

ERKPNACKDE (SEQ ID NO: 20, an exemplary GuaB epitope amino acid sequence, such as a heterologous epitope where the backbone protein is derived from IpaD)

CTTCCGGAGTATCGGGGAGAGGATGGTGTCAGAGTG (SEQ ID NO: 21, an exemplary nucleic acid sequence encoding a Stx2 epitope, such as a heterologous epitope where the backbone protein is derived from IpaD)

LPEYRGEDGVRV (SEQ ID NO: 22, an exemplary Stx2 epitope amino acid sequence, such as a heterologous epitope where the backbone protein is derived from IpaD)

GAGTATACAAAATATAATGATGACGATACC (SEQ ID NO: 23, an exemplary nucleic acid sequence encoding a StxB epitope, such as a heterologous epitope where the backbone protein is derived from IpaD)

EYTKYNDDDT (SEQ ID NO: 24, an exemplary StxB epitope amino acid sequence, such as a heterologous epitope where the backbone protein is derived from IpaD).

### Exemplary Fusion Proteins

In specific examples, the fusion protein can include a backbone protein derived from IpaD that includes one or more epitopes (for example, 1, 2, 3, 4, 5, 6, 7, 8, 9, 10, or more epitopes) from Shigella virulence factors IpaD, IpaB, VirG, GuaB, StxA, Stx2A, and/or StxB. Example sequences include the following:

(SEQ ID NO: 1
ATGGGCAATATAACAACTCTGACTAATAGTATTTCCACCTCATCATTCA
GCCCAAACAATACCAACGGTTCATCAACCGAAACAGTTAATTCTGATAT
AAAAACAACGACCAGTTCTCATCCTGTAAGTTCCCTTACTATGCTCAAC
GACACCCTTCATAATATCAGAACAACAAATCAGGCATTAAAGAAAGATC
TCTCACAAAAAACGTTGACTAAAACATCGCTAGAAGAAATAGCATTACA
TTCATCTCAGATTAGCATGGATGTAAACAAATCCGCTCAACTATTGGAT
ATTCTTTCCAAGAAAGAATATCCAATTAATAAAGACGCAAGAGAATTAT
TACATTCAGCCCAACTAGTTGGAAAAAATAATGAAGAATCTATGATATC
TCACAGAGAACTGTGGGATAAAATTGCAAAGTCAATCAATAATATTAAT
GAACAGTATCTGAAAGTATATGAACATGCCGTTAGTTCATATACTCAAT
CTGCTGAACAGCTATCAACCCAGCAGAAAAGTCTTGCCGGCTGGATCTC
TCCCGGAGGTAACGACGGAAACTCCGTGAAATTACAAGTCAAGTCTCTT
AAAAGTGATAGTGATGGGGGGAATGGAGGTGATAAAGATAAACCGCTAT
ATCCAGCAAATAATACTGTTCTGCCTGACTATCATGGACAAGACTCTGT
TCGTGTAGAATTAGGTGGAACAGAACGTAAACCGAACGCTTGTAAAGAC
GAGTATGTTGTCAATATAAACATGACCCCAATAGACAATATGCTTCCGG
AGTATCGGGGAGAGGATGGTGTCAGAGTGGGTGAGGTTGTGCTAGATAA
TGCAAAATATCAGGCATGGAATGCCGGAGAGTATACAAAATATAATGAT
GACGATACCAATCTTCAAACCTTAGTTCAAAAATACAGTAATGCCAATA
GTATTTTTGATAATTTAGTAAAGGTTTTGAGTAGTACAATAAGTTCATG
TACAGATACAGATAAACTTTTTCTCCATTTCTGA,

an exemplary nucleic acid sequence encoding a fusion protein with a Shigella IpaD backbone protein and homologous and heterologous epitopes from Shigella virulence factors IpaD, IpaB, VirG, GuaB, StxA, Stx2A, and StxB (epitopes underlined))

(SEQ ID NO: 2
MGNITTLTNSISTSSFSPNNINGSSTETVNSDIKTTTSSHPVSSLTMLN
DTLHNIRTTNQALKKDLSQKTLTKTSLEEIALHSSQISMDVNKSAQLLD
ILSKKEYPINKDARELLHSAQLVGKNNEESMISHRELWDKIAKSINNIN
EQYLKVYEHAVSSYTQSAEQLSTQQKSLAGWISPGGNDGNSVKLQVKSL
KSDSDGGNGGDKDKPLYPANNTVLPDYHGQDSVRVELGGTERKPNACKD
EYVVNINMTPIDNMLPEYRGEDGVRVGEVVLDNAKYQAWNAGEYTKYND
DDTNLQTLVQKYSNANSIFDNLVKVLSSTISSCTDTDKLFLHF,

an exemplary fusion protein with a Shigella IpaD backbone protein and homologous and heterologous epitopes from Shigella virulence factors IpaD, IpaB, VirG, GuaB, StxA, Stx2A, and StxB (epitopes underlined)).

Proteins, peptides, and nucleic acids that are similar to those disclosed herein can be used as well as fragments thereof that retain biological activity. These proteins, peptides, and nucleic acids may contain variations, substitutions, deletions, or additions. In some examples, the differences can be in regions not significantly conserved among different species. Such regions can be identified by aligning the amino acid sequences of related proteins, peptides, and nucleic acids from various species. Generally, the biological effects of a molecule are retained, for example, immunogenicity or ability to elicit an immune response in a subject. For example, a protein, peptide, and/or nucleic acid at least 75%, 80%, 85%, 90%, 95%, 96%, 97%, 98% or 99% identical to one of these molecules can be utilized. Proteins or peptides (or nucleic acids encoding such proteins or peptides) may include at most 1, 2, 3, 4, 5, 6, 7, 8, 9, or 10 conservative amino acid substitutions. Generally, modified proteins or peptides (or nucleic acids encoding such proteins or peptides) retain at least 75%, 80%, 85%, 90%, 95%, 96%, 97%, 98% or 99% of the biological function of the native molecule or have increased biological function as compared to the native molecule.

Also included are derivatives or modifications of the backbone proteins, epitopes (such as heterologous or homologous epitopes), or fusion proteins, which are differentially modified during or after synthesis, such as by benzylation, glycosylation, acetylation, phosphorylation, amidation, pegylation, derivatization by known protecting/blocking groups. In some embodiments, peptides can include at least one amino acid or every amino acid that is a D stereoisomer. Other peptides can include at least one amino acid that is reversed. The amino acid that is reversed may be a D stereoisomer. Every amino acid of a peptide may be reversed and/or every amino acid can be a D stereoisomer.

Other fusion proteins can be utilized in combination with the fusion proteins disclosed herein, such as fusion proteins that include ETEC and/or toxoid antigens (for example, as disclosed in Int. Pub. No. WO 2015/095335 A1 and Zhang et al., PLoS One 10(3):e0121623, 2015, both of which are incorporated herein by reference in their entireties). Exemplary sequences include the following:

SEQ ID NO: 25,
ATGGAAATGGCTAGCGCAGTAGAGGATTTTTTCATTGTTCCAGTTTCTG
GAGATCCTGCAATTGATCTTTTGCAAGCTGATGGCAATGCTCTGCCATC
AGCTGTAAAGTTAGCTTATTCTCCCGCATCAAAAACTAATACTTTGGTG
GGTGTTTTGACTCTTGTACATACAAACGATGCAACTAAAAAAAATGTAC
TAGTTAAGCTTGTAACACCACAGCTTACAGATGTTCTGAATCCAACCCT
GCAAATTCCTGTTTCTGTGCAGGTAACGGTCTACCCTGTTTCTACAACA
GCCAAAGAATTTGAAGCTGCTGCTTTGGGATATTCTGCATCCGGTGTAA
ATGGCTTGGTGTCAATTGTGCTTACTGTAATTAGCGCTGCACCTAAAAC
TGCCGGTACCGCCCCAACTGCAGGAAACTATTCAGGAGTAGTATCTCTT
GTAATGACTTTGGGAGCCTGATAACGGCCGCACTCGAGC
(CFA/I/II/IV MEFA,

an exemplary nucleic acid encoding a fusion protein with ETEC antigens, such as displayed by epitopes, for example, as encoded by the underlined nucleotides)

SEQ ID NO: 26,
MEMASAVEDFFIVPVSGDPAIDLLQADGNALPSAVKLAYSPASKTNTLV
GVLTLVHTNDATKKNVLVKLVTPQLTDVLNPTLQIPVSVQVTVYPVSTT
AKEFEAAALGYSASGVNGLVSIVLTVISAAPKTAGTAPTAGNYSGVVSL
VMTLGA (CFA/I/II/IV MEFA,

an exemplary fusion protein with ETEC antigens, such as displayed by epitopes, for example, as indicated by the underlined nucleotides)

SEQ ID NO: 27,
ATGGAAATGGCTAGCATGAATAGTAGCAATTACTGCTGTGAATTGTGTT
GTAGCCCTGCTTGTACCGGGTGCTATGGGCCGGGGCCCAATGGCGACAA
ATTATACCGTGCTGACTCTAGACCCCCAGATGAAATAAAACGTTCCGGA
GGTCTTATGCCCAGAGGGCATAATGAGTACTTCGATAGAGGAACTCAAA
TGAATATTAATCTTTATGATCACGCGAGAGGAACACAAACCGGCTTTGT
CAGATATGATGACGGATATGTTTCCACTTCTCTTAGTTTGAGAAGTGCT
CACTTAGCAGGACAGTCTATATTATCAGGATATTCCACTTACTATATAT
ATGTTATAGCGACAGCACCAAATATGTTTAATGTTAATGATGTATTAGG
CGTATACAGCCCTCACCCATATGAACAGGAGGTTTCTGCGTTAGGTGGA
ATACCATATTCTCAGATATATGGATGGTATCGTGTTAATTTTGGTGTGA
TTGATGAACGATTACATCGTAACAGGGAATATAGAGACCGGTATTACAG
AAATCTGAATATAGCTCCGGCAGAGGATGGTTACAGATTAGCAGGTTTC
CCACCGGATCACCAAGCTTGGAGAGAAGAACCCTGGATTCATCATGCAC
CACAAGGTTGTGGAAATTCATCAGGAGGGCCGGTCGACATGAATAGTAG
CAATTACTGCTGTGAATTGTGTTGTAGCCCTGCTTGTACCGGGTGCTAT
ACAATTACAGGTGATACTTGTAATGAGGAGACCCAGAATCTGAGCACAA
TATATGCCAGGAAATATCAATCAAAAGTTAAGAGGCAGATATTTTCAGA
CTATCAGTCAGAGGTTGACATATATAACAGAATTCGGAATGAATTAGGG
CCGGGGCCCGCTCCCCAGTCTATTACAGAACTATGTTCGGAATATCGCA
ACACACAAATATATACGATAAATGACAAGATACTATCATATACGGAATC
GATGGCAGGCAAAAGAGAAATGGTTATCATTACATTTAAGAGCGGCGCA
ACATTTCAGGTCGAAGTCCCGGGCAGTCAACATATAGACTCCCAAAAAA
AAGCCATTGAAAGGATGAAGGACACATTAAGAATCACATATCTGACCGA
GACCAAAATTGATAAATTATGTGTATGGAATAATAAAACCCCCAATTCA
ATTGCGGCAATCAGTGATCCCCGGGTACCGAGCTCGATGAATAGTAGCA
ATTACTGCTGTGAATTGTGTTGTAGCCCTGCTTGTACCGGGTGCTATTA
ATAA (toxoid fusion 3xSTa_{N12S}-mnLT_{R192G/L211A},

an exemplary nucleic acid encoding a fusion protein with ETEC antigens, such as displayed by epitopes, for example, as encoded by the underlined nucleotides)

SEQ ID NO: 28,
MEMASMNSSNYCCELCCSPACTGCYGPGPNGDKLYRADSRPPDEIKRSG
GLMPRGHNEYFDRGTQMNINLYDHARGTQTGFVRYDDGYVSTSLSLRSA
HLAGQSILSGYSTYYIYVIATAPNMFNVNDVLGVYSPHPYEQEVSALGG
IPYSQIYGWYRVNFGVIDERLHRNREYRDRYYRNLNIAPAEDGYRLAGF
PPDHQAWREEPWIHHAPQGCGNSSGGPVDMNSSNYCCELCCSPACTGCY
TITGDTCNEETQNLSTIYARKYQSKVKRQIFSDYQSEVDIYNRIRNELG
PGPAPQSITELCSEYRNTQIYTINDKILSYTESMAGKREMVIITFKSGA
TFQVEVPGSQHIDSQKKAIERMKDTLRITYLTETKIDKLCVWNNKTPNS
IAAISDPRVPSSMNSSNYCCELCCSPACTGCY
(toxoid fusion 3xST_{aN12S}-mnLT_{R192G/L211A},

an exemplary fusion protein with ETEC antigens, such as displayed by epitopes, for example, as indicated by the underlined nucleotides).

Any of the disclosed backbone proteins, epitopes, or fusion proteins can be readily synthesized by automated solid phase procedures known in the art. Techniques and procedures for solid phase synthesis are described in Solid Phase Peptide Synthesis: A Practical Approach, by E. Atherton and R. C. Sheppard, published by IRL, Oxford University Press, 1989. Alternatively, these proteins, epitopes, or fusion proteins may be prepared by way of segment condensation, as described, for example, in Liu et al., Tetrahedron Lett. 37:933-936, 1996; Baca et al., J. Am. Chem. Soc. 117:1881-1887, 1995; Tam et al., Int. J. Peptide Protein Res. 45:209-216, 1995; Schnolzer and Kent, Science 256:221-225, 1992; Liu and Tam, J. Am. Chem. Soc. 116:4149-4153, 1994; Liu and Tam, Proc. Natl. Acad. Sci. USA 91:6584-6588, 1994; and Yamashiro and Li, Int. J. Peptide Protein Res. 31:322-334, 1988). Other methods useful for synthesizing peptides of the present disclosure are described in Nakagawa et al., J. Am. Chem. Soc. 107:7087-7092, 1985.

### Nucleic Acids and Host Cells

Nucleic acids encoding the fusion proteins disclosed herein are also provided. These nucleic acids include DNA, cDNA and RNA sequences which encode the fusion protein, for example, including the nucleic acid sequences disclosed herein. The coding sequence includes variants that result from the degeneracy (e.g., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3rd Edition, W.H. 5 Freeman and Co., NY).

A nucleic acid encoding the fusion protein can be cloned or amplified by in vitro methods, such as the polymerase chain reaction (PCR), the ligase chain reaction (LCR), the transcription-based amplification system (TAS), the self-sustained sequence replication system (3 SR) and the Qβreplicase amplification system (QB). For example, a polynucleotide encoding the protein can be isolated by polymerase chain reaction of cDNA using primers based on the DNA sequence of the molecule. A wide variety of cloning and in vitro amplification methodologies are well-known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, N Y, 1989). Polynucleotides also can be isolated by screening genomic or cDNA libraries with probes selected from the sequences of the desired polynucleotide under stringent hybridization conditions.

A polynucleotide sequence encoding the fusion protein can be operatively linked to expression control sequences. An expression control sequence operatively linked to a coding sequence is linked such that expression of the coding sequence is achieved under conditions compatible with the expression control sequences. The expression control sequences include, but are not limited to, appropriate promoters, enhancers, a ribosome binding site, transcription terminators, transcriptional regulators (e.g., AraC and Lad), a start codon (e.g., ATG) in front of a protein-encoding gene, splicing signal for introns, maintenance of the correct reading frame of that gene to permit proper translation of mRNA, and/or stop codons.

The polynucleotides encoding the fusion protein include a recombinant DNA, which is incorporated into a vector such as an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

In some embodiments, vectors are used for fusion protein expression. A person of skill in the art will understand that there are many options for protein expression systems, including systems that express protein in yeast, insect, and bacterial cells. In specific, non-limiting examples, E. coli cells can be used. In examples, protein expression systems can include vectors with one or more tags for purification of the fusion protein, such as histidine (His), chitin-binding protein (CBP), maltose-binding protein (MBP), or glutathione-S-transferase (GST), or a streptavidin tag. In specific, non-limiting examples, a vector with a His tag, such as a pET28a vector.

In another embodiment, vectors are used for expression in yeast, such as Saccharomyces cerevisiae or Kluyveromyces lactis. Several promoters are known to be of use in yeast expression systems such as the constitutive promoters plasma membrane H⁺-ATPase (PMAI), glyceraldehyde-3-phosphate dehydrogenase (GPD), phosphoglycerate kinase-1 (PGK1), alcohol dehydrogenase-1 (ADH1), and pleiotropic drug-resistant pump (PDR5). In addition, many inducible promoters are of use, such as GAL1-10 (induced by galactose), PHO5 (induced by low extracellular inorganic phosphate), and tandem heat shock HSE elements (induced by temperature elevation to 37° C.). Promoters that direct variable expression in response to a titratable inducer include the methionine-responsive MET3 and MET25 promoters and copper-dependent CUP1 promoters. Any of these promoters may be cloned into multicopy (20 or single copy (CEN) plasmids to give an additional level of control in expression level. The plasmids can include nutritional markers (such as URA3, ADE3, HIS1, and others) for selection in yeast and antibiotic resistance (such as AMP) for propagation in bacteria. Plasmids for expression on K. lactis are known, such as pKLAC1. Thus, in one example, after amplification in bacteria, plasmids can be introduced into the corresponding yeast auxotrophs by methods similar to bacterial transformation. The polynucleotides can also be designed to express in insect cells.

The fusion protein can be expressed in a variety of yeast strains. For example, seven pleiotropic drug-resistant transporters, YOR1, SNQ2, PDR5, YCF1, PDR10, PDR11, and PDR15, together with their activating transcription factors, PDR1 and PDR3, have been simultaneously deleted in yeast host cells, rendering the resultant strain sensitive to drugs. Yeast strains with altered lipid composition of the plasma membrane, such as the erg6 mutant defective in ergosterol biosynthesis, can also be utilized. Proteins that are highly sensitive to proteolysis can be expressed in a yeast lacking the master vacuolar endopeptidase Pep4, which controls the activation of other vacuolar hydrolases. Heterologous expression in strains carrying temperature-sensitive (ts) alleles of genes can be employed if the corresponding null mutant is inviable.

Viral vectors can also be prepared encoding the fusion protein disclosed herein. A number of viral vectors have been constructed, including polyoma, SV40 (Madzak et al., J. Gen. Virol., 73:15331536, 1992), adenovirus (Berkner, Cur. Top. Microbiol. Immunol., 158:39-6, 1992; Berliner et al., Bio Techniques, 6:616-629, 1998; Gorziglia et al., J. Virol., 66:4407-4412, 1992; Quantin et al., Proc. Natl. Acad. Sci. USA, 89:2581-2584, 1992; Rosenfeld et al., Cell, 68:143-155, 1992; Wilkinson et al., Nucl. Acids Res., 20:2233-2239, 1992; Stratford-Perricaudet et al., Hum. Gene Ther., 1:241-256, 1990), vaccinia virus (Mackett et al., Biotechnology, 24:495-499, 1992), adeno-associated virus (Muzyczka, Curr. Top. Microbiol. Immunol., 158:91-123, 1992; On et al., Gene, 89:279-282, 1990), herpes viruses including HSV and EBV (Margolskee, Curr. Top. Microbiol. Immunol., 158:67-90, 1992; Johnson et al., J. Virol., 66:29522965, 1992; Fink et al., Hum. Gene Ther. 3:11-19, 1992; Breakfield et al., Mol. Neurobiol., 1:337-371, 1978; Fresse et al., Biochem. Pharmacol., 40:2189-2199, 1990), Sindbis viruses (H. Herweijer et al., Human Gene Therapy, 6:1161-1167, 1995; U.S. Pat. Nos. 5,091,309 and 5,2217,879), alphaviruses (S. Schlesinger, Trends Biotechnol. 11:18-22, 1993; I. Frolov et al., Proc. Natl. Acad. Sci. USA, 93:11371-11377, 1996) and retroviruses of avian (Brandyopadhyay et al., Mol. Cell Biol., 4:749-754, 1984; Petropouplos et al., J. Virol., 66:3391-3397, 1992), murine (Miller, Curr. Top. Microbiol. Immunol., 158:1-24, 1992; Miller et al., Mol. Cell Biol., 5:431-437, 1985; Sorge et al., Mol. Cell Biol., 4:1730-1737, 1984; Mann et al., J. Virol., 54:401-407, 1985), and human origin (Page et al., J. Virol., 64:5370-5276, 1990; Buchschalcher et al., J. Virol., 66:2731-2739, 1992). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

Thus, in one embodiment, the polynucleotide encoding a fusion protein is included in a viral vector. Suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors, and poliovirus vectors.

DNA sequences encoding the fusion protein can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Hosts cells also can include microbial, insect, and mammalian host cells. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, Jakoby and Pastan (eds), Methods in Enzymology: Cell Culture, volume 58, Academic Press, Inc., Harcourt Brace Jovanovich, N.Y., 1979). Examples of commonly used mammalian host cell lines are VERO and HeLa cells, CHO cells, and WI38, BHK, and COS cell lines, although cell lines may be used, such as cells designed to provide higher expression desirable glycosylation patterns, or other features. Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be co-transformed with polynucleotide sequences encoding a C-terminal endostatin polypeptide, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see, for example, Eukaryotic Viral Vectors, Cold Spring Harbor Laboratory, Gluzman ed., 1982).

### Chemical Conjugation

While molecular methods can be used to synthesize fusion proteins, chemical methods can alternatively be used to link a peptide to a backbone polypeptide. In some examples, peptides from a backbone protein (such as IpaD) can be covalently bound to epitope(s) (such as epitopes from IpaD, IpaB, VirG, GuaB, StxA, Stx2A, and/or StxB) through chemical conjugation. Various types of chemical reagents can be used (see, e.g., Ido et al., JBC, 287(31): 26377-26387, 2012, and U.S. Pat. Pub. No. 2003/0040496, both incorporated herein by reference). In some examples, 1-ethyl-3-(3-dimethylaminopropyl)carbodiimide hydrochloride (EDC) and/or N-hydroxysulfosuccinimide (Sulfo-NHS) is used for chemical conjugation (see, e.g., Ido et al., JBC, 287(31): 26377-26387, 2012, incorporated herein by reference)

In other embodiments, alternative chemical conjugation (e.g., cross-linking) reagents may be used to form covalent bonds between amino groups and thiol groups and to introduce thiol groups into proteins (see, e.g., U.S. Pat. Pub. No. 2003/0040496, incorporated herein by reference). Additional alternative chemical conjugation (e.g., cross-linking) reagents can be found in the PIERCE CATALOG, ImmunoTechnology Catalog & Handbook, 1992-1993, which describes the preparation of and use of such reagents and provides a commercial source for such reagents (incorporated herein by reference; see also, e.g., Cumber et al., Bioconjugate Chem. 3:397-401, 1992; Thorpe et al., Cancer Res. 47:5924-5931, 1987; Gordon et al., Proc. Natl. Acad Sci. 84:308-312, 1987; Walden et al., J. Mol. Cell Immunol. 2:191-197, 1986; Carlsson et al., Biochem. J. 173:723-737, 1978; Mahan et al., Anal. Biochem., 162:163-170, 1987; Wawryznaczak et al., Br. J. Cancer 66:361-366, 1992; Fattom et al., Infection & Immun. 60:584-589, 1992, all of which are incorporated herein by reference). These reagents include, but are not limited to: N-succinimidyl-3-(2-pyridyldithio)propionate (SPDP; disulfide linker); sulfosuccinimidyl 6-[3-(2-pyridyldithio)propionamido]hexanoate (sulfo-LC-SPDP); succinimidyloxycarbonyl-α-methyl benzyl thiosulfate (SMBT, hindered disulfate linker); succinimidyl 6-[3-(2-pyridyldithio) propionamido]hexanoate (LC-SPDP); sulfosuccinimidyl 4-(N-maleimidomethyl)cyclohexane-1-carboxylate (sulfo-SMCC); succinimidyl 3-(2-pyridyldithio)butyrate (SPDB; hindered disulfide bond linker); sulfosuccinimidyl 2-(7-azido-4-methylcoumarin-3-acetamide) ethyl-1,3′-dithiopropionate (SAED); sulfosuccinimidyl 7-azido-4-methylcoumarin-3-acetate (SAMCA); sulfosuccinimidyl 6-[alpha-methyl-alpha-(2-pyridyldithio)toluamido]hexanoate (sulfo-LC-SMPT); 1,4-di-[3′-(2′-pyridyldithio)propionamido]butane (DPDPB); 4-succinimidyloxycarbonyl-ethyl-(2-pyridylthio)toluene (SMPT, hindered disulfate linker); sulfosuccinimidyl6[-methyl-2-pyridyldithio)toluamido]hexanoate (sulfo-LC-SMPT); m-maleimidobenzoyl-N-hydroxysuccinimide ester (MB S); m-maleimidobenzoyl-N-hydroxysulfosuccinimide ester (sulfo-MB S); N-succinimidyl(4-iodoacetyl)aminobenzoate (SIAB; thioether linker); sulfosuccinimidyl(4-iodoacetyl)amino benzoate (sulfo-SLAB); succinimidyl4(p-maleimidophenyl)butyrate (SMPB); sulfosuccinimidyl-4-(p-maleimidophenyl)butyrate (sulfo-SMPB); and azidobenzoyl hydrazide (ABH).

### Pharmaceutical Compositions

Pharmaceutical compositions provided herein include a fusion protein or nucleic acid as disclosed and a pharmaceutically acceptable carrier. Such compositions can be administered to subjects by a variety of administration modes known to the person of ordinary skill in the art, for example, intramuscular, subcutaneous, intravenous, intra-arterial, intra-articular, intraperitoneal, intradermal, or parenteral routes. In specific examples, the compositions can be administered via subcutaneous, intradermal, or intramuscular routes. Actual methods for preparing administrable compositions will be known or apparent to those skilled in the art and are described in more detail in such publications as Remington's Pharmaceutical Sciences, by E. W. Martin, Mack Publishing Co., Easton, PA, 22nd Edition, 2013.

Fusion proteins or nucleic acids described herein can be formulated with pharmaceutically acceptable carriers to help retain biological activity while also promoting increased stability during storage within an acceptable temperature range. Potential carriers include, but are not limited to, physiologically balanced culture medium, phosphate buffered saline solution, water, emulsions (for example, oil/water or water/oil emulsions), various types of wetting agents, cryoprotective additives or stabilizers such as proteins, peptides or hydrolysates (for example, albumin, gelatin), sugars (for example, sucrose, lactose, sorbitol), amino acids (for example, sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The compositions of the disclosure can contain as pharmaceutically acceptable carriers substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The disclosed compositions may optionally include an adjuvant to enhance an immune response of the host. Adjuvants, such as aluminum hydroxide (ALHYDROGEL®, available from Brenntag Biosector, Copenhagen, Denmark and Amphogel®, Wyeth Laboratories, Madison, NJ), Freund's adjuvant, MPL™ (3-O-deacylated monophosphoryl lipid A; Corixa, Hamilton, IN), IL-12 (Genetics Institute, Cambridge, MA), TLR agonists (such as TLR-9 agonists), among many other suitable adjuvants, can be included in the compositions. Suitable adjuvants are, for example, toll-like receptor agonists, alum, A1PO4, alhydrogel, Lipid-A and derivatives or variants thereof, dmLT, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), among many other suitable adjuvants well known in the art, may be used as an adjuvant (Newman et al., 1998, Critical Reviews in Therapeutic Drug Carrier Systems 15:89-142). These adjuvants help to stimulate the immune system in a non-specific way, thus enhancing the immune response to a pharmaceutical product. In some embodiments, the adjuvant is selected to elicit a Th1 biased immune response in a subject.

In some instances, the adjuvant formulation includes a mineral salt, such as a calcium or aluminum (alum) salt, for example calcium phosphate, aluminum phosphate or aluminum hydroxide. In some embodiments, the adjuvant includes an oil and water emulsion, for example, an oil-in-water emulsion (such as MF59 (Novartis) or AS03 (GlaxoSmithKline). One example of an oil-in-water emulsion comprises a metabolizable oil, such as squalene, a tocol such as a tocopherol, for example, alpha-tocopherol, and a surfactant, such as sorbitan trioleate (Span 85) or polyoxyethylene sorbitan monooleate (Tween 80), in an aqueous carrier.

In some instances, it may be desirable to combine a disclosed composition with other pharmaceutical products (for example, vaccines, such as Ty21a, or other fusion proteins, such as including ETEC and/or toxoid antigens, for example, ETEC antigens CFA/I/II/IV MEFA, SEQ ID NO: 26, and/or toxoid fusion 3xSTa_{N12S}-mnLT_{R192G/L211A}, SEQ ID NO: 28), which induce protective responses to other agents. For example, a composition including a fusion protein or nucleic acid as described herein can be can be administered simultaneously or sequentially with other vaccines recommended by the Advisory Committee on Immunization Practices (ACIP; cdc.gov/vaccines/acip/index) for the targeted age group (for example, children at or less than 5 years old, children at or less than one year old, children over 5 years old, or adults). As such, a disclosed composition described herein may be administered simultaneously or sequentially with vaccines against, for example, ETEC, typhoid (for example, Ty21a), measles virus, rubella virus, varicella zoster virus, hepatitis B (HepB), diphtheria, tetanus and pertussis (DTaP), pneumococcal bacteria (PCV), Haemophilus influenzae type b (Hib), polio, influenza, and/or rotavirus.

In some embodiments, the composition can be provided as a sterile composition. The composition typically contains an effective amount of a disclosed fusion protein or nucleic acid encoding the fusion protein. Typically, the amount of fusion protein or nucleic acid in each dose of the composition is selected as an amount which induces an immune response without significant, adverse side effects. In some embodiments, the composition can be provided in unit dosage form for use to induce an immune response in a subject, for example, to prevent or inhibit Shigella or E. coli infection in the subject. A unit dosage form contains a suitable single preselected dosage for administration to a subject, suitable marked or measured multiples of two or more preselected unit dosages, and/or a metering mechanism for administering the unit dose or multiples thereof.

Methods

In some embodiments, methods of inducing an immune response to Shigella in a subject are provided. The disclosed methods include administering one or more compositions disclosed herein to a subject. In some examples, the methods induce an immune response to Shigella (such as one or more of S. flexneri, S. sonnei, S. dysenteriae, and/or S. boydii) or E. coli (such as enterotoxigenic E. coli) in a subject. In some embodiments, the subject is a human. The immune response can be a protective immune response, for example, a response that inhibits or reduces subsequent infection by Shigella (such as S. flexneri, S. sonnei, S. dysenteriae, and/or S. boydii) or E. coli (such as enterotoxigenic E. coli). Eliciting the immune response can also be used to treat or inhibit Shigella (such as S. flexneri, S. sonnei, S. dysenteriae, and/or S. boydii) or E. coli (such as enterotoxigenic E. coli) infection and illnesses associated therewith.

In some examples, a subject is selected for treatment that has, or is at risk for developing Shigella (such as S. flexneri, S. sonnei, S. dysenteriae, and/or S. boydii) or E. coli (such as enterotoxigenic E. coli) infection, for example, due to exposure or the possibility of exposure to Shigella (such as S. flexneri, S. sonnei, S. dysenteriae, and/or S. boydii) or E. coli (such as enterotoxigenic E. coli). Following administration of a disclosed composition, the subject can be monitored for development of antibodies to Shigella (such as S. flexneri, S. sonnei, S. dysenteriae, and/or S. boydii) or E. coli (such as enterotoxigenic E. coli), infection, symptoms associated therewith, or a combination thereof.

Typical subjects for administration or treatment with the compositions and methods of the present disclosure include humans and any other animals susceptible to infection by Shigella (such as S. flexneri, S. sonnei, S. dysenteriae, and/or S. boydii) or E. coli (such as enterotoxigenic E. coli). The compositions can be administered, for example, by beginning an immunization regimen anytime from about 6 months to 12 months of age, or from about 12 months to 15 months of age, or from about 2 years to 5 years of age, or from about 4 years to 6 years of age. In particular examples, a child is administered a first dose at 12-15 months of age and a second dose between 4-6 years of age. Booster doses at later ages can also be administered, such as if it is determined that the subject exhibits waning immunity against Shigella (such as S. flexneri, S. sonnei, S. dysenteriae, and/or S. boydii) or E. coli (such as enterotoxigenic E. coli) infection. In other examples, the compositions are administered to a subject prior to travel to an area where Shigella and/or E. coli is endemic, for example 1 to 12 months (such as 1 to 3 months, 2 to 6 months, 4 to 8 months, or 8 to 12 months) prior to travel. Booster doses can also be administered prior to travel.

Administration of a disclosed fusion protein or nucleic acid can be for prophylactic or therapeutic purpose. When provided prophylactically, the fusion protein or nucleic acid can be provided in advance of any symptom, for example, in advance of infection. The prophylactic administration serves to inhibit or ameliorate any subsequent infection. In some embodiments, the methods can involve selecting a subject at risk for contracting Shigella (such as S. flexneri, S. sonnei, S. dysenteriae, and/or S. boydii) or E. coli (such as enterotoxigenic E. coli) infection and administering a therapeutically effective amount of a disclosed fusion protein or nucleic acid to the subject. The fusion protein or nucleic acid can be provided prior to the anticipated exposure to Shigella (such as S. flexneri, S. sonnei, S. dysenteriae, and/or S. boydii) or E. coli (such as enterotoxigenic E. coli) so as to attenuate the anticipated severity, duration, or extent of an infection and/or associated disease symptoms, after exposure or suspected exposure to the pathogen, or after the actual initiation of an infection. When provided therapeutically, the disclosed fusion proteins or nucleic acids are provided at or after the onset of a symptom of Shigella (such as S. flexneri, S. sonnei, S. dysenteriae, and/or S. boydii) or E. coli (such as enterotoxigenic E. coli) infection or after diagnosis of Shigella (such as S. flexneri, S. sonnei, S. dysenteriae, and/or S. boydii) or E. coli (such as enterotoxigenic E. coli) infection.

In some embodiments, administration of a disclosed fusion protein or nucleic acid to a subject can elicit the production of an immune response that is protective against or reduces the severity of symptoms or complications of Shigella (such as S. flexneri, S. sonnei, S. dysenteriae, and/or S. boydii) or E. coli (such as enterotoxigenic E. coli) infection, such as encephalitis or meningitis, when the subject is subsequently infected or re-infected with Shigella (such as S. flexneri, S. sonnei, S. dysenteriae, and/or S. boydii) or E. coli (such as enterotoxigenic E. coli). While the naturally circulating pathogen may still be capable of causing infection, there can be a reduced possibility of symptoms as a result of the vaccination and a possible increase in resistance to subsequent infection by Shigella (such as S. flexneri, S. sonnei, S. dysenteriae, and/or S. boydii) or E. coli (such as enterotoxigenic E. coli).

The fusion proteins and nucleic acids described herein as well as compositions thereof are provided to a subject in an amount effective to induce or enhance an immune response against Shigella (such as S. flexneri, S. sonnei, S. dysenteriae, and/or S. boydii) or E. coli (such as enterotoxigenic E. coli) in the subject, such as a human. The actual dosage of disclosed fusion protein or nucleic acid will vary according to factors, such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimized or improved response.

A composition including one or more of the disclosed fusion proteins or nucleic acids can be used in coordinate (or prime-boost) vaccination protocols or combinatorial formulations. In certain embodiments, novel combinatorial immunogenic compositions and coordinate immunization protocols employ separate immunogens or formulations, each directed toward eliciting an anti-pathogen immune response, such as an immune response to proteins from Shigella (such as S. flexneri, S. sonnei, S. dysenteriae, and/or S. boydii) or E. coli (such as enterotoxigenic E. coli). In specific examples, a combinatorial immunogenic composition can include any of the immunogenic compositions disclosed herein (such as a fusion protein including a backbone protein and at least one heterologous epitope or nucleic acids encoding a fusion protein) and an additional fusion protein. In specific examples, the additional fusion protein includes enterotoxigenic E. coli (ETEC) antigens CFA/I/II/IV MEFA and/or toxoid fusion 3xSTa_{N12S}-mnLT_{R192G/L211A}.

Separate immunogenic compositions that elicit an anti-pathogen immune response can be combined in a polyvalent immunogenic composition administered to a subject in a single immunization step, or they can be administered separately (in monovalent immunogenic compositions) in a coordinate (or prime-boost) immunization protocol.

There can be several boosts, and each boost can be the same or a different disclosed immunogen (such as a fusion protein including the same epitopes as the initial administration or a fusion protein including one or more different epitopes as the initial administration). In some examples, the boost may be the same fusion protein as another boost or the prime. The prime and boost can be administered as a single dose or multiple doses, for example two doses, three doses, four doses, five doses, six doses, or more can be administered to a subject over days, weeks, or months. Multiple boosts can also be given, such one to five (for example, 1, 2, 3, 4, or 5 boosts) or more. Different dosages can be used in a series of sequential immunizations. For example a relatively large dose can be used in a primary immunization and then a boost with relatively smaller doses.

In some embodiments, the boost can be administered about two, about three to eight, or about four, weeks following the prime, or several months after the prime. In some embodiments, the boost can be administered about 5, about 6, about 7, about 8, about 10, about 12, about 18, about 24, about 36, about 48, or about 50 months after the prime, or more or less time after the prime. Periodic additional boosts can also be used at appropriate time points to enhance the subject's “immune memory.” The adequacy of the vaccination parameters chosen, for example, formulation, dose, regimen and the like, can be determined by taking aliquots of serum from the subject and assaying antibody titers during the course of the immunization program. In addition, the clinical condition of the subject can be monitored for the desired effect, for example, prevention of Shigella (such as S. flexneri, S. sonnei, S. dysenteriae, and/or S. boydii) or E. coli (such as enterotoxigenic E. coli) infection or improvement in disease state (for example, reduction in pathogen load). If such monitoring indicates that vaccination is sub-optimal, the subject can be boosted with one or more additional doses, and the administration parameters can be modified in a fashion expected to potentiate the immune response.

The amount of disclosed fusion protein, nucleic acids, or composition thereof administered can vary. In some embodiments, the amount administered ranges from about 1-100, 5-50, 1-10, 1-20, 5-15, 5-25, 5-30, 10-50, 10-60, or 25-75 μg/dose (such as via IM, ID, or SC). The amount utilized in an immunogenic composition is selected based on the subject population (for example, infant or elderly). The amount for a particular composition can be ascertained by standard studies involving observation of antibody titers and other responses in subjects. It is understood that a therapeutically effective amount of a disclosed fusion protein, nucleic acids, or composition thereof, can include an amount that is ineffective at eliciting an immune response by administration of a single dose, but that is effective upon administration of multiple dosages, for example, in a prime-boost administration protocol.

Upon administration of a disclosed composition, the immune system of the subject typically responds to the immunogenic composition by producing antibodies specific for a pathogenic protein. Such a response signifies that an immunologically effective dose was delivered to the subject.

For each particular subject, specific dosage regimens can be evaluated and adjusted over time according to the individual need and professional judgment of the person administering or supervising the administration of the immunogenic composition. The dosage and number of doses will depend on the setting, for example, in an adult or anyone primed by prior Shigella (such as S. flexneri, S. sonnei, S. dysenteriae, and/or S. boydii) or E. coli (such as enterotoxigenic E. coli) infection or immunization, a single dose may be a sufficient booster. In naïve subjects, in some examples, at least two doses are administered for example, at least two or three doses.

In some embodiments, the antibody response of a subject is determined in the context of evaluating effective dosages/immunization protocols. In most instances, it is sufficient to assess the antibody titer in serum or plasma obtained from the subject. Decisions as to whether to administer booster inoculations and/or to change the amount of the therapeutic agent administered to the individual can be at least partially based on the antibody titer level. The antibody titer level can be based on, for example, an immunobinding assay which measures the concentration of antibodies in the serum which bind to a Shigella or E. coli protein.

Determination of effective dosages is typically based on animal model studies followed up by human clinical trials and is guided by administration protocols that significantly reduce the occurrence or severity of targeted disease symptoms or conditions in the subject, or that induce a desired response in the subject (such as a neutralizing immune response). Suitable models in this regard include, for example, murine, rat, porcine, feline, ferret, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of the composition (for example, amounts that are effective to elicit a desired immune response or alleviate one or more symptoms of a targeted disease). In alternative embodiments, an effective amount or effective dose of the composition may simply inhibit or enhance one or more selected biological activities correlated with a disease or condition, as set forth herein, for either therapeutic or diagnostic purposes.

Administration of a composition that elicits an immune response to reduce or prevent a Shigella (such as S. flexneri, S. sonnei, S. dysenteriae, and/or S. boydii) or E. coli (such as enterotoxigenic E. coli) infection, can, but does not necessarily completely, eliminate such an infection, so long as the infection is measurably diminished. For example, administration of an effective amount of the composition can decrease the Shigella (such as S. flexneri, S. sonnei, S. dysenteriae, and/or S. boydii) or E. coli (such as enterotoxigenic E. coli) infection (for example, as measured by infection of cells or by number or percentage of subjects infected by Shigella (such as S. flexneri, S. sonnei, S. dysenteriae, and/or S. boydii) or E. coli (such as enterotoxigenic E. coli)) by a desired amount, for example, by at least 10%, at least 20%, at least 50%, at least 60%, at least 70%, at least 80%, at least 90%, at least 95%, at least 98%, or even at least 99%, as compared to a suitable control.

In some embodiments, administration of a therapeutically effective amount of one or more of the disclosed fusion proteins or nucleic acids to a subject induces a neutralizing immune response in the subject. To assess neutralization activity, following immunization of a subject, serum can be collected from the subject at appropriate time points, frozen, and stored for neutralization testing. Methods to assay for neutralization activity are known to the person of ordinary skill in the art, and include, but are not limited to microneutralization assays, flow cytometry-based assays, and single-cycle infection assays.

In certain embodiments, the fusion protein or nucleic acid can be administered sequentially with other anti-Shigella or E. coli therapeutic agents (such as an antibiotic), such as before or after the other agent. One of ordinary skill in the art would know that sequential administration can mean immediately following or after an appropriate period of time, such as hours, days, weeks, months, or even years later.

### EXAMPLES

The following examples are provided to illustrate certain particular features and/or embodiments. These examples should not be construed to limit the disclosure to the particular features or embodiments described.

### Example 1

Shigella Multi-Epitope Fusion Antigen Protein

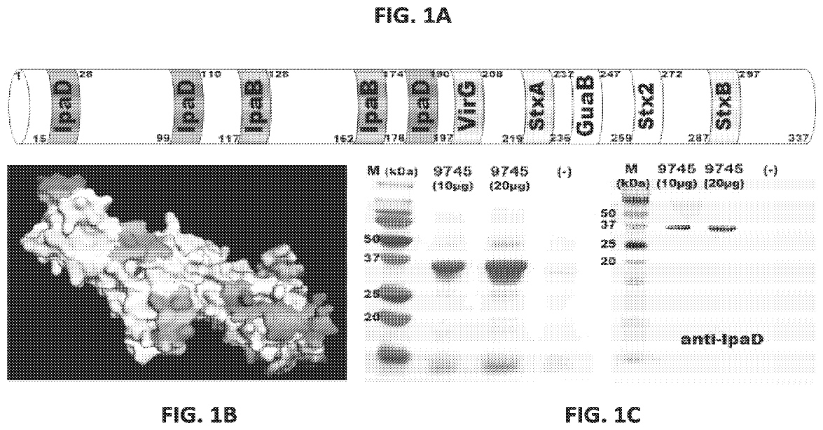

Shigella MEFA with virulence factor epitopes exposed were constructed and expressed. B-cell immunodominant epitopes from backbone IpaD (SPNNTNGSSTET (SEQ ID NO: 6), SKKEYPINKD (SEQ ID NO: 8), SPGGNDGNSV (SEQ ID NO: 14)), IpaB (QLVGKNNEES (SEQ ID NO: 10), SAEQLSTQQKS (SEQ ID NO: 12)), VirG (SDSDGGNGGD (SEQ ID NO: 16)), GuaB (ERKPNACKDE (SEQ ID NO: 20)), Shiga toxin A subunit (LPDYHGQDSVRV (SEQ ID NO: 18)), Stx2 A subunit (LPEYRGEDGVRV (SEQ ID NO: 22)), and Shiga toxin B subunit (EYTKYNDDDT (SEQ ID NO: 24)) were in silico identified and selected for the construction of Shigella MEFA protein (FIG. 1A). Amino acid sequence alignment showed that these epitopes were identical or highly conserved across the Shigella species and serotypes examined. All epitopes were exposed on the surface of Shigella MEFA protein (FIG. 1B) and predicted to be immunogenic. The Shigella MEFA protein (37.2 kDa) was expressed by E. coli strain BL21, and was extracted at a yield of 110 mg per liter culture broth and with a purity estimated of greater than 90% based on Coomassie blue staining; extracted protein after refolding was recognized by anti-IpaD and anti-IpaB antisera (FIG. 1C).

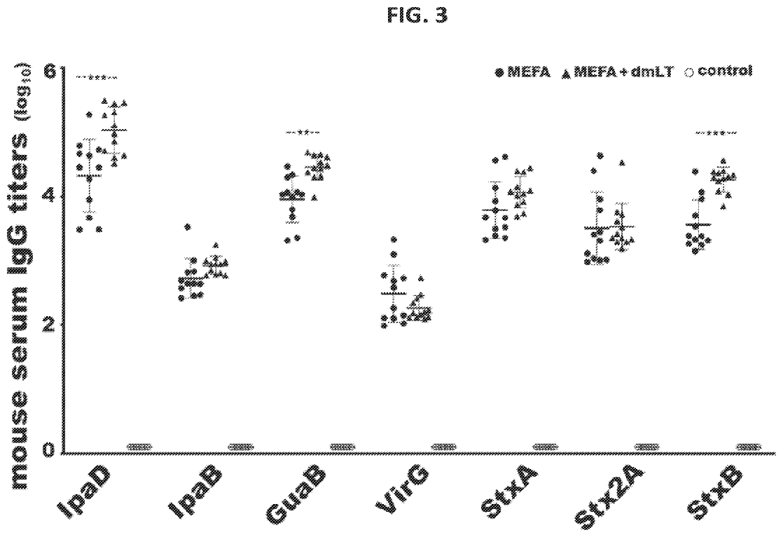

Shigella MEFA protein was broadly immunogenic when administered intramuscularly in mice. Adult mice were intramuscularly (IM) immunized with Shigella MEFA protein, with or without adjuvant dmLT, and developed robust immune responses to IpaB, IpaD, VirG, GuaB, StxA, Stx2A and StxB (FIG. 3). The IgG titers in the serum samples of the mice IM immunized with Shigella MEFA protein (without adjuvant) were 2.7±0.30, 4.3±0.57, 2.5±0.45, 4.0±0.36, 3.8±0.44, 3.5±0.57, and 3.6±0.39 (log 10) to IpaB, IpaD, VirG, GuaB, StxA, Stx2A and StxB, respectively. When dmLT adjuvant was included, the antigen-specific IgG titers were detected at 2.9±0.14, 5.0±0.36, 2.3±0.19, 4.5±0.20, 4.1±0.25, 3.5±0.36, and 4.3±0.19 (log₁₀) IpaB, IpaD, VirG, GuaB, StxA, Stx2A and StxB, respectively, from the sera of the immunized mice. The IgG titers to IpaD, GuaB and StxB were greater in the group immunized with dmLT adjuvant. No antigen-specific IgG responses were detected from the control mice.

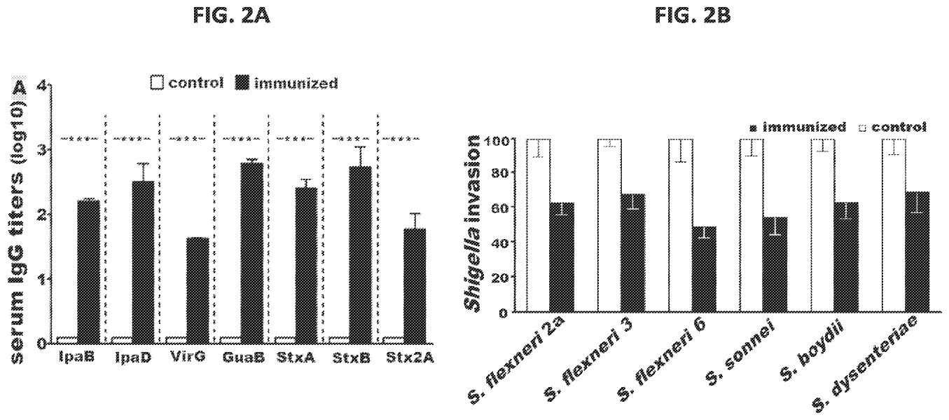

Shigella MEFA-induced antibodies significantly inhibited invasion of Shigella spp. and serotypes in vitro. The serum samples pooled from the mice immunized with Shigella MEFA, with or without dmLT, significantly inhibited the invasion of S. flexneri serotypes 2a, 3 and 6, S. sonnei, S. boydii and S. dysenteriae type 1 to HeLa cells (FIG. 2B). S. flexneri serotypes 2a, 3 and 6, S. sonnei, S. boydii, and S. dysenteriae type 1 bacteria incubated with the serum of the mice immunized with Shigella MEFA showed 25.6% to 51% reduction at invading to HeLa cells, compared to the bacteria treated with the control mouse serum samples. Antibody invasion inhibition activities from the serum samples between the two immunization groups, with or without adjuvant dmLT, were not significantly different against the four species and six serotypes.

### Example 2

Combination of Shigella MEFA with Enterotoxigenic E. coli MEFA

Because Shigella MEFA is a single protein, it can be combined with other proteins, for example, adhesin and toxin MEFA proteins of enterotoxigenic E. coli (ETEC) or cholera MEFA (Vibrio cholerae), to develop combination vaccines against Shigella and ETEC or cholera. This Shigella MEFA can also be expressed by a licensed vaccine strain, for example, Ty21a, which is a vaccine for typhoid fever, generating new vaccines for two types of pathogens and diseases.

Similar to Shigella, ETEC is also a leading cause of children's diarrhea and travelers' diarrhea. A combination vaccine is desirable because Shigella and ETEC infect the same populations (children and international travelers). An ETEC-Shigella combination vaccine could reduce manufacturing cost, simplify transportation and storage logistics, and fit a single EPI (expanded program on immunization) schedule.

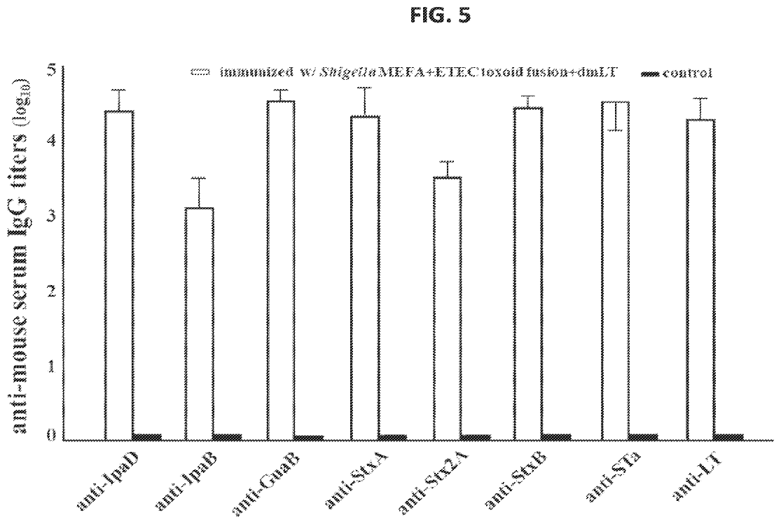

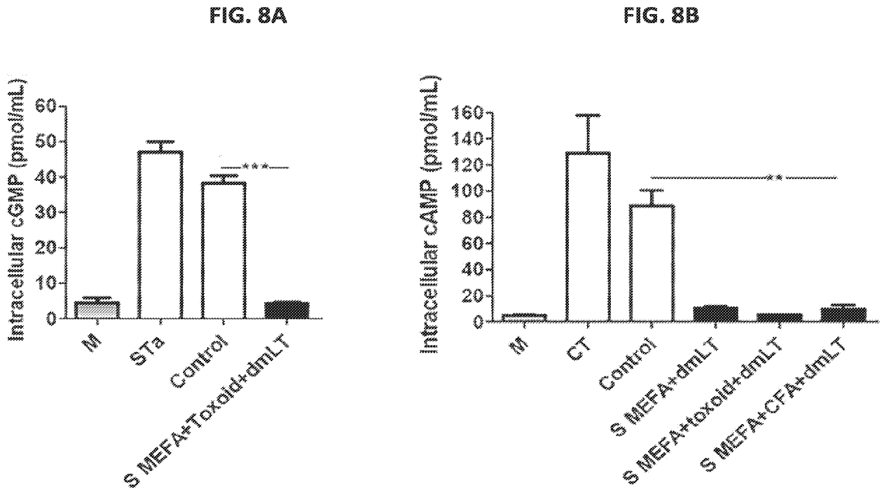

This Shigella MEFA is antigenically compatible with ETEC antigens CFA/I/II/IV MEFA and toxoid fusion 3xSTa_{N12S}-mnLT_{R192G/L211A}, See Ruan et al., Clin. Vaccine Immunol., 21(2):243-9, 2014, and Ruan et al., Infect. Immun., 82(5):1823-1832, 2014. Mice that were IM immunized with Shigella MEFA and ETEC toxoid fusion 3xSTa_{N12S}-mnLT_{R192G/L211A} developed robust Shigella and ETEC antigen-specific antibody responses (FIG. 5). Furthermore, the induced antibodies neutralized enterotoxicity of both ETEC toxins (LT and STa) (FIGS. 8A-8B).

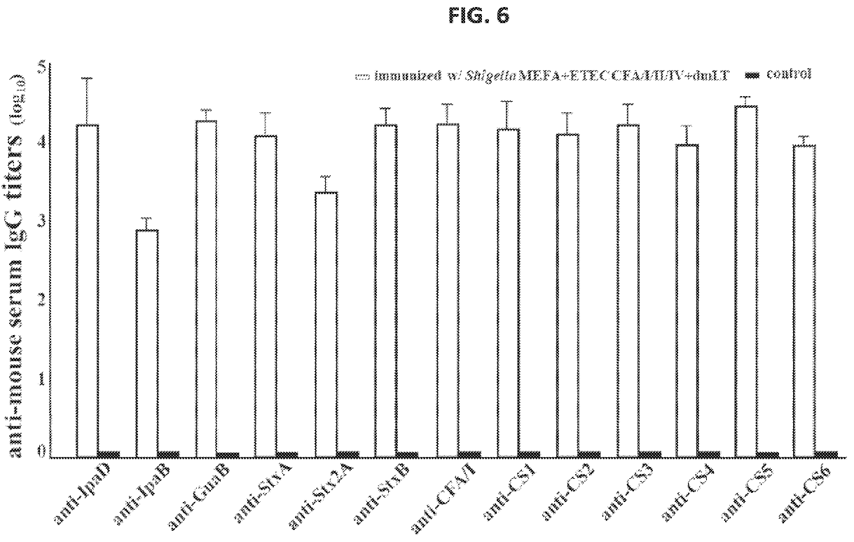

Shigella MEFA can also be co-administered with ETEC adhesin MEFA CFA/I/II/IV to induce broad antibody responses to Shigella antigens and all seven ETEC adhesins (FIG. 6). Moreover, the derived anti-mouse serum antibodies inhibited adherence of ETEC bacteria expressing any one or two of the seven adhesins.

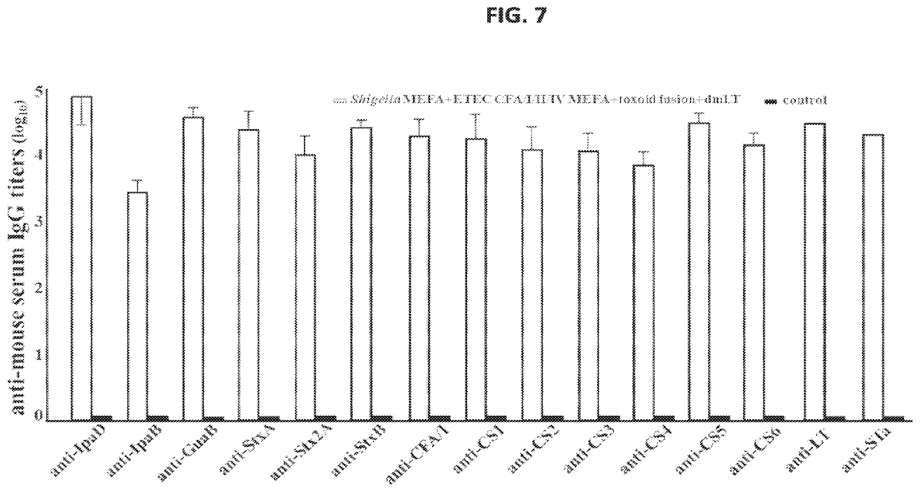

Moreover, Shigella MEFA can be combined with two ETEC antigens (CFA/I/II/IV MEFA and toxoid fusion 3xSTa_{N12S}-mnLT_{R192G/L211A}). When IM co-administered, three antigens induced antigen-specific antibody responses to all target antigens (FIG. 7). All the immunized mice developed strong IgG antibodies to Shigella IpaB, IpaD, GuaB, StxA, Stx2A, and StxB (VirG is undergoing) as well as to ETEC CFA/I, CS1, CS2, CS3, CS4, CS5, CS6, LT, and STa.

In view of the many possible embodiments to which the principles of the disclosed subject matter may be applied, it should be recognized that the illustrated embodiments are only examples of the disclosure and should not be taken as limiting the scope of the disclosure. Rather, the scope of the disclosure is defined by the following claims.

1. A fusion protein, comprising a backbone protein and at least one heterologous epitope, wherein the backbone protein comprises a consensus sequence with at least 90% identity to SEQ ID NO: 4.

2-5. (canceled)

6. The fusion protein of claim 1, wherein the backbone protein comprises SEQ ID NO: 4.

7. The fusion protein of claim 1, wherein the at least one heterologous epitope comprises a peptide of a Shigella virulence factor.

8. The fusion protein of claim 7, wherein the Shigella virulence factor comprises one or more of IpaB, VirG, GuaB, StxA, Stx2A, and StxB.

9. The fusion protein of claim 7, wherein the at least one heterologous epitope comprises one or more of SEQ ID NOs: 10, 12, 16, 18, 20, 22, and 24.

10. The fusion protein of claim 1, further comprising at least one homologous epitope.

11. The fusion protein of claim 10, wherein the homologous epitope comprises one or more of SEQ ID NOs: 6, 8, and 14.

12. The fusion protein of claim 11, wherein
the at least one heterologous epitope comprises each of SEQ ID NOs: 10, 12, 16, 18, 20, 22, and 24; and
the at least one homologous epitope comprises each of SEQ ID NOs: 6, 8, and 14.

13. The fusion protein of claim 12, comprising an amino acid sequence having at least 90% identity to SEQ ID NO: 2, or wherein the fusion protein comprises SEQ ID NO: 2.

14. A nucleic acid encoding the fusion protein of claim 1.

15. The nucleic acid of claim 14, wherein the nucleic acid is at least 90% identical to SEQ ID NO: 3, or the nucleic acid comprises SEQ ID NO: 3.

16-21. (canceled)

22. The nucleic acid of claim 14, wherein the nucleic acid is at least 90% identical to SEQ ID NO: 1, or the nucleic acid comprises SEQ ID NO: 1.

23. A vector, comprising the nucleic acid of claim 14.

24-28. (canceled)

29. A pharmaceutical composition comprising a pharmaceutically acceptable carrier and the fusion protein of claim 1, or a nucleic acid encoding the fusion protein.

30. The pharmaceutical composition of claim 29, further comprising an adjuvant.

31. (canceled)

32. The pharmaceutical composition of claim 29, further comprising an additional fusion protein.

33. The pharmaceutical composition of claim 32, wherein the additional fusion protein comprises at least one peptide of Escherichia coli or Vibrio cholera.

34. The pharmaceutical composition of claim 29, wherein the fusion protein, or the nucleic acid encoding the fusion protein, is expressed in Ty21a.

35. A method of inducing an immune response to Shigella in a subject, comprising administering to the subject an effective amount of the pharmaceutical composition of claim 29.

36. The method of claim 35, further comprising administering an adjuvant.

37. The method of claim 35, wherein the fusion protein, or the nucleic acid encoding the fusion protein, is expressed in Ty21a, and the method comprises administering the Ty21a expressing the fusion protein or nucleic acid to the subject.

38-40. (canceled)

41. The method of claim 35, wherein the pharmaceutical composition is administered subcutaneously (SC), intramuscularly (IM), intradermally (ID), or orally.

42. (canceled)

43. The method of claim 35, wherein the subject is human.


SEQUENCE LISTING (28 sequences)

SEQ ID NO: 1 - DNA, 1014 bp, Artificial Sequence
  A MEFA encoding nucleic acid
atgggcaatataacaactctgactaatagtatttccacctcatcattcagcccaaacaataccaacggttcatcaaccgaaacagttaattctgatataaaaacaacgaccagttctcatcctgtaagttcccttactatgctcaacgacacccttcataatatcagaacaacaaatcaggcattaaagaaagatctctcacaaaaaacgttgactaaaacatcgctagaagaaatagcattacattcatctcagattagcatggatgtaaacaaatccgctcaactattggatattctttccaagaaagaatatccaattaataaagacgcaagagaattattacattcagcccaactagttggaaaaaataatgaagaatctatgatatctcacagagaactgtgggataaaattgcaaagtcaatcaataatattaatgaacagtatctgaaagtatatgaacatgccgttagttcatatactcaatctgctgaacagctatcaacccagcagaaaagtcttgccggctggatctctcccggaggtaacgacggaaactccgtgaaattacaagtcaagtctcttaaaagtgatagtgatggggggaatggaggtgataaagataaaccgctatatccagcaaataatactgttctgcctgactatcatggacaagactctgttcgtgtagaattaggtggaacagaacgtaaaccgaacgcttgtaaagacgagtatgttgtcaatataaacatgaccccaatagacaatatgcttccggagtatcggggagaggatggtgtcagagtgggtgaggttgtgctagataatgcaaaatatcaggcatggaatgccggagagtatacaaaatataatgatgacgataccaatcttcaaaccttagttcaaaaatacagtaatgccaatagtatttttgataatttagtaaaggttttgagtagtacaataagttcatgtacagatacagataaactttttctccatttctga

SEQ ID NO: 2 - protein, 337 aa, Artificial Sequence
  A MEFA protein
MGNITTLTNSISTSSFSPNNTNGSSTETVNSDIKTTTSSHPVSSLTMLNDTLHNIRTTNQALKKDLSQKTLTKTSLEEIALHSSQISMDVNKSAQLLDILSKKEYPINKDARELLHSAQLVGKNNEESMISHRELWDKIAKSINNINEQYLKVYEHAVSSYTQSAEQLSTQQKSLAGWISPGGNDGNSVKLQVKSLKSDSDGGNGGDKDKPLYPANNTVLPDYHGQDSVRVELGGTERKPNACKDEYVVNINMTPIDNMLPEYRGEDGVRVGEVVLDNAKYQAWNAGEYTKYNDDDTNLQTLVQKYSNANSIFDNLVKVLSSTISSCTDTDKLFLHF

SEQ ID NO: 3 - DNA, 1014 bp, Artificial Sequence
  A backbone encoding nucleic acid
  misc_feature (49)..(84): ′N′ indicates any nucleic acid
  misc_feature (301)..(330): ′N′ indicates any nucleic acid
  misc_feature (355)..(384): ′N′ indicates any nucleic acid
  misc_feature (490)..(522): ′N′ indicates any nucleic acid
  misc_feature (538)..(567): ′N′ indicates any nucleic acid
  misc_feature (592)..(621): ′N′ indicates any nucleic acid
  misc_feature (658)..(693): ′N′ indicates any nucleic acid
  misc_feature (709)..(738): ′N′ indicates any nucleic acid
  misc_feature (778)..(813): ′N′ indicates any nucleic acid
  misc_feature (862)..(891): ′N′ indicates any nucleic acid
atgggcaatataacaactctgactaatagtatttccacctcatcattcnnnnnnnnnnnnnnnnnnnnnnnnnnnnnnnnnnnngttaattctgatataaaaacaacgaccagttctcatcctgtaagttcccttactatgctcaacgacacccttcataatatcagaacaacaaatcaggcattaaagaaagatctctcacaaaaaacgttgactaaaacatcgctagaagaaatagcattacattcatctcagattagcatggatgtaaacaaatccgctcaactattggatattcttnnnnnnnnnnnnnnnnnnnnnnnnnnnnnngcaagagaattattacattcagccnnnnnnnnnnnnnnnnnnnnnnnnnnnnnnatgatatctcacagagaactgtgggataaaattgcaaagtcaatcaataatattaatgaacagtatctgaaagtatatgaacatgccgttagttcatatactcaannnnnnnnnnnnnnnnnnnnnnnnnnnnnnnnncttgccggctggatcnnnnnnnnnnnnnnnnnnnnnnnnnnnnnnaaattacaagtcaagtctcttaaannnnnnnnnnnnnnnnnnnnnnnnnnnnnnaaagataaaccgctatatccagcaaataatactgttnnnnnnnnnnnnnnnnnnnnnnnnnnnnnnnnnnnngaattaggtggaacannnnnnnnnnnnnnnnnnnnnnnnnnnnnntatgttgtcaatataaacatgaccccaatagacaatatgnnnnnnnnnnnnnnnnnnnnnnnnnnnnnnnnnnnnggtgaggttgtgctagataatgcaaaatatcaggcatggaatgccggannnnnnnnnnnnnnnnnnnnnnnnnnnnnnaatcttcaaaccttagttcaaaaatacagtaatgccaatagtatttttgataatttagtaaaggttttgagtagtacaataagttcatgtacagatacagataaactttttctccatttctga

SEQ ID NO: 4 - protein, 337 aa, Artificial Sequence
  A backbone protein
  MISC_FEATURE (17)..(28): ′Xaa′ indicates any amino acid
  MISC_FEATURE (101)..(110): ′Xaa′ indicates any amino acid
  MISC_FEATURE (119)..(128): ′Xaa′ indicates any amino acid
  MISC_FEATURE (164)..(174): ′Xaa′ indicates any amino acid
  MISC_FEATURE (180)..(189): ′Xaa′ indicates any amino acid
  MISC_FEATURE (198)..(207): ′Xaa′ indicates any amino acid
  MISC_FEATURE (220)..(231): ′Xaa′ indicates any amino acid
  MISC_FEATURE (237)..(246): ′Xaa′ indicates any amino acid
  MISC_FEATURE (260)..(271): ′Xaa′ indicates any amino acid
  MISC_FEATURE (288)..(297): ′Xaa′ indicates any amino acid
MGNITTLTNSISTSSFXXXXXXXXXXXXVNSDIKTTTSSHPVSSLTMLNDTLHNIRTTNQALKKDLSQKTLTKTSLEEIALHSSQISMDVNKSAQLLDILXXXXXXXXXXARELLHSAXXXXXXXXXXMISHRELWDKIAKSINNINEQYLKVYEHAVSSYTQXXXXXXXXXXXLAGWIXXXXXXXXXXKLQVKSLKXXXXXXXXXXKDKPLYPANNTVXXXXXXXXXXXXELGGTXXXXXXXXXXYVVNINMTPIDNMXXXXXXXXXXXXGEVVLDNAKYQAWNAGXXXXXXXXXXNLQTLVQKYSNANSIFDNLVKVLSSTISSCTDTDKLFLHF

SEQ ID NO: 5 - DNA, 36 bp, Artificial Sequence
  An IpaD epitope encoding nucleic acid
agcccaaacaataccaacggttcatcaaccgaaaca

SEQ ID NO: 6 - protein, 12 aa, Artificial Sequence
  An IpaD epitope
SPNNTNGSSTET

SEQ ID NO: 7 - DNA, 30 bp, Artificial Sequence
  An IpaD epitope encoding nucleic acid
tccaagaaagaatatccaattaataaagac

SEQ ID NO: 8 - protein, 10 aa, Artificial Sequence
  An IpaD epitope
SKKEYPINKD

SEQ ID NO: 9 - DNA, 30 bp, Artificial Sequence
  An IpaB epitope encoding nucleic acid
caactagttggaaaaaataatgaagaatct

SEQ ID NO: 10 - protein, 10 aa, Artificial Sequence
  An IpaB epitope
QLVGKNNEES

SEQ ID NO: 11 - DNA, 33 bp, Artificial Sequence
  An IpaB epitope encoding nucleic acid
tctgctgaacagctatcaacccagcagaaaagt

SEQ ID NO: 12 - protein, 11 aa, Artificial Sequence
  An IpaB epitope
SAEQLSTQQKS

SEQ ID NO: 13 - DNA, 30 bp, Artificial Sequence
  An IpaD epitope encoding nucleic acid
tctcccggaggtaacgacggaaactccgtg

SEQ ID NO: 14 - protein, 10 aa, Artificial Sequence
  An IpaD epitope
SPGGNDGNSV

SEQ ID NO: 15 - DNA, 30 bp, Artificial Sequence
  A VirG epitope encoding nucleic acid
agtgatagtgatggggggaatggaggtgat

SEQ ID NO: 16 - protein, 10 aa, Artificial Sequence
  A VirG epitope
SDSDGGNGGD

SEQ ID NO: 17 - DNA, 36 bp, Artificial Sequence
  A StxA epitope encoding nucleic acid
ctgcctgactatcatggacaagactctgttcgtgta

SEQ ID NO: 18 - protein, 12 aa, Artificial Sequence
  A StxA epitope
LPDYHGQDSVRV

SEQ ID NO: 19 - DNA, 30 bp, Artificial Sequence
  A GuaB epitope encoding nucleic acid
gaacgtaaaccgaacgcttgtaaagacgag

SEQ ID NO: 20 - protein, 10 aa, Artificial Sequence
  A GuaB epitope
ERKPNACKDE

SEQ ID NO: 21 - DNA, 36 bp, Artificial Sequence
  A Stx2 epitope encoding nucleic acid
cttccggagtatcggggagaggatggtgtcagagtg

SEQ ID NO: 22 - protein, 12 aa, Artificial Sequence
  A Stx2 epitope
LPEYRGEDGVRV

SEQ ID NO: 23 - DNA, 30 bp, Artificial Sequence
  A StxB epitope encoding nucleic acid
gagtatacaaaatataatgatgacgatacc

SEQ ID NO: 24 - protein, 10 aa, Artificial Sequence
  A StxB epitope
EYTKYNDDDT

SEQ ID NO: 25 - DNA, 480 bp, Artificial Sequence
  CFA/I/II/IV MEFA encoding nucleic acid
atggaaatggctagcgcagtagaggattttttcattgttccagtttctggagatcctgcaattgatcttttgcaagctgatggcaatgctctgccatcagctgtaaagttagcttattctcccgcatcaaaaactaatactttggtgggtgttttgactcttgtacatacaaacgatgcaactaaaaaaaatgtactagttaagcttgtaacaccacagcttacagatgttctgaatccaaccctgcaaattcctgtttctgtgcaggtaacggtctaccctgtttctacaacagccaaagaatttgaagctgctgctttgggatattctgcatccggtgtaaatggcttggtgtcaattgtgcttactgtaattagcgctgcacctaaaactgccggtaccgccccaactgcaggaaactattcaggagtagtatctcttgtaatgactttgggagcctgataacggccgcactcgagc

SEQ ID NO: 26 - protein, 153 aa, Artificial Sequence
  CFA/I/II/IV MEFA protein
MEMASAVEDFFIVPVSGDPAIDLLQADGNALPSAVKLAYSPASKTNTLVGVLTLVHTNDATKKNVLVKLVTPQLTDVLNPTLQIPVSVQVTVYPVSTTAKEFEAAALGYSASGVNGLVSIVLTVISAAPKTAGTAPTAGNYSGVVSLVMTLGA

SEQ ID NO: 27 - DNA, 1278 bp, Artificial Sequence
  Toxoid fusion 3xSTaN12S-mnLTR192G/L211A encoding nucleic acid
atggaaatggctagcatgaatagtagcaattactgctgtgaattgtgttgtagccctgcttgtaccgggtgctatgggccggggcccaatggcgacaaattataccgtgctgactctagacccccagatgaaataaaacgttccggaggtcttatgcccagagggcataatgagtacttcgatagaggaactcaaatgaatattaatctttatgatcacgcgagaggaacacaaaccggctttgtcagatatgatgacggatatgtttccacttctcttagtttgagaagtgctcacttagcaggacagtctatattatcaggatattccacttactatatatatgttatagcgacagcaccaaatatgtttaatgttaatgatgtattaggcgtatacagccctcacccatatgaacaggaggtttctgcgttaggtggaataccatattctcagatatatggatggtatcgtgttaattttggtgtgattgatgaacgattacatcgtaacagggaatatagagaccggtattacagaaatctgaatatagctccggcagaggatggttacagattagcaggtttcccaccggatcaccaagcttggagagaagaaccctggattcatcatgcaccacaaggttgtggaaattcatcaggagggccggtcgacatgaatagtagcaattactgctgtgaattgtgttgtagccctgcttgtaccgggtgctatacaattacaggtgatacttgtaatgaggagacccagaatctgagcacaatatatgccaggaaatatcaatcaaaagttaagaggcagatattttcagactatcagtcagaggttgacatatataacagaattcggaatgaattagggccggggcccgctccccagtctattacagaactatgttcggaatatcgcaacacacaaatatatacgataaatgacaagatactatcatatacggaatcgatggcaggcaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagaccaaaattgataaattatgtgtatggaataataaaacccccaattcaattgcggcaatcagtgatccccgggtaccgagctcgatgaatagtagcaattactgctgtgaattgtgttgtagccctgcttgtaccgggtgctattaataa

SEQ ID NO: 28 - protein, 424 aa, Artificial Sequence
  Toxoid fusion 3xSTaN12S-mnLTR192G/L211A protein
MEMASMNSSNYCCELCCSPACTGCYGPGPNGDKLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSGGPVDMNSSNYCCELCCSPACTGCYTITGDTCNEETQNLSTIYARKYQSKVKRQIFSDYQSEVDIYNRIRNELGPGPAPQSITELCSEYRNTQIYTINDKILSYTESMAGKREMVIITFKSGATFQVEVPGSQHIDSQKKAIERMKDTLRITYLTETKIDKLCVWNNKTPNSIAAISDPRVPSSMNSSNYCCELCCSPACTGCY